(19) United States
(12) Patent Application Publication
(10) Pub. No.: US 2022/0401673 A1
(43) Pub. Date: Dec. 22, 2022

(54) SYSTEMS AND METHODS FOR INJECTING SUBSTANCES INTO A RESPIRATORY SYSTEM

(71) Applicant: ResMed Sensor Technologies Limited, Dublin (IE)

(72) Inventors: Redmond Shouldice, Dublin (IE); Stephen McMahon, Dublin (IE)

(21) Appl. No.: 17/773,007

(22) PCT Filed: Oct. 30, 2020
(86) PCT No.: PCT/IB2020/060237

§ 371 Date: Apr. 28, 2022

Related U.S. Application Data
(60) Provisional application No. 62/929,095, filed on Oct. 31, 2019.
(60) Provisional application No. 62/953,332, filed on Dec. 24, 2019.

(51) Int. Cl. A61M 16/00; A61M 16/16

(52) U.S. Cl. CPC A61M 16/024; A61M 16/16; A61M 2205/3653; A61M 2205/42

(57) ABSTRACT

A method for delivering a substance into an airway of a user via a respiratory system comprises receiving the substance within a receptacle; receiving physiological data; determining one or more sleep-related parameters based on the physiological data; and modifying the delivery of the substance into the airway of the user based at least in part on the one or more sleep-related parameters. The respiratory system includes a respiratory device configured to supply pressurized air to the airway of the user via a conduit and a user interface. The respiratory device, the user interface, and the conduit form an air pathway. The respiratory device is configured to include or engage the receptacle such that an outlet of the receptacle is in direct or indirect fluid communication with the air pathway.

### CROSS-REFERENCE TO RELATED APPLICATIONS

This application claims the benefit of, and priority to, U.S. Provisional Patent Application No. 62/929,095 filed on Oct. 31, 2019 and U.S. Provisional Patent Application No. 62/953,332 filed on Dec. 24, 2019, each of which is hereby incorporated by reference herein in its entirety.

### TECHNICAL FIELD

The present disclosure relates generally to systems and methods for delivering medicine to a user, and more particularly, to systems and methods for delivering medicine to a user through an air pathway of a respiratory system.

### BACKGROUND

Various systems exist for aiding users experiencing sleep apnea and related respiratory disorders. A range of respiratory disorders exist that can impact users. Certain disorders are characterized by particular events (e.g., apneas, hypopneas, hyperpneas, or any combination thereof). Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), and Chest wall disorders. At least some of these events and disorders can be treated or at least ameliorated with medicine. Thus, a need exists for systems and methods for delivering medicine to the user utilizing the air pathway of the respiratory system. The present disclosure is directed to solving these and other problems.

### SUMMARY

According to some implementations of the present disclosure, a method for delivering a substance into an airway of a user via a respiratory system comprises receiving the substance within a receptacle; receiving physiological data; determining one or more sleep-related parameters based on the physiological data; and modifying the delivery of the substance into the airway of the user based at least in part on the one or more sleep-related parameters.

According to some implementations of the present disclosure, a method for delivering a substance into an airway of a user via a respiratory system comprises receiving the substance within a receptacle; receiving physiological data; determining a current sleep stage of the user based on the physiological data, other data, or both; and modifying the delivery of the substance into the airway of the user based at least in part on the current sleep stage of the user.

According to some implementations of the present disclosure, a method for delivering a substance into an airway of a user via a respiratory system comprises receiving the substance within a receptacle; receiving physiological data; determining whether the user has experienced a predetermined event based on the physiological data; and modifying the delivery of the substance into the airway of the user based at least in part on the predetermined event.

According to some implementations of the present disclosure, a method for delivering a substance into an airway of a user via a respiratory system comprises receiving the substance within a receptacle; receiving physiological data based on exhaled breath of the user; determining the effectiveness of the substance based on the physiological data; and modifying the delivery of the substance into the airway of the user based at least in part on the determined effectiveness of the substance.

According to some implementations, a respiratory therapy system comprises a respiratory device and a receptacle. The respiratory device is configured to supply pressurized air to an airway of a user via a user interface coupled to the respiratory device via a conduit. The respiratory device, the user interface, and the conduit form an air pathway. The receptacle includes a substance therein, and is fluidly coupled to the air pathway so that the substance can be delivered into the air pathway.

The above summary is not intended to represent each embodiment or every aspect of the present invention. Additional features and benefits of the present invention are apparent from the detailed description and figures set forth below.

### BRIEF DESCRIPTION OF THE DRAWINGS

FIG. 1 is a functional block diagram for a system for delivering a substance into an airway of a user, according to some implementations of the present disclosure;

FIG. 2 is a perspective view of the system of FIG. 1, a user of the system, and a bed partner of the user, according to some implementations of the present disclosure;

FIG. 3 illustrates an exemplary timeline for a sleep session, according to some implementations of the present disclosure;

FIG. 4 illustrates an exemplary hypnogram associated with the sleep session of FIG. 3, according to some implementations of the present disclosure;

FIG. 5A is a rear perspective view of a respiratory device of the system of FIG. 1, according to some implementations of the present disclosure;

FIG. 5B is a rear perspective view of the respiratory device of FIG. 5A showing an injection plug fluidly coupled between the respiratory device and a conduit, according to some implementations of the present disclosure;

FIG. 5C is a rear perspective view of the respiratory device of FIG. 5A showing the alignment between the injection plug of FIG. 5B and the conduit, according to some implementations of the present disclosure;

FIG. 6A is a perspective view of a first implementation of the injection plug of FIG. 5B, according to some implementations of the present disclosure;

FIG. 6B is a cross-sectional view of the first implementation of the injection plug of FIG. 5B, according to some implementations of the present disclosure;

FIG. 7A is a perspective view of a second implementation of the injection plug of FIG. 5B, according to some implementations of the present disclosure;

FIG. 7B is a cross-sectional view of the second implementation of the injection plug of FIG. 5B, according to some implementations of the present disclosure;

FIG. 8A is a rear perspective view of a respiratory device of the system of FIG. 1 showing a cover controlling access to a receptacle disposed in the respiratory device, according to some implementations of the present disclosure;

FIG. 8B is a partially transparent rear perspective view of the respiratory device of FIG. 8A including the receptacle and a blower motor therein, according to some implementations of the present disclosure;

FIG. 9 is a process flow diagram for a first method of delivering a substance into an airway of the user, according to some implementations of the present disclosure;

FIG. 10 is a process flow diagram for a second method of delivering a substance into an airway of the user, according to some implementations of the present disclosure;

FIG. 11 is a process flow diagram for a third method of delivering a substance into an airway of the user, according to some implementations of the present disclosure; and

FIG. 12 is a process flow diagram for a fourth method of delivering a substance into an airway of the user, according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders. Examples of sleep-related and/or respiratory disorders include Periodic Limb Movement Disorder (PLMD), Restless Leg Syndrome (RLS), Sleep-Disordered Breathing (SDB), Obstructive Sleep Apnea (OSA), Central Sleep Apnea (CSA), other types of apneas, Cheyne-Stokes Respiration (CSR), respiratory insufficiency, Obesity Hyperventilation Syndrome (OHS), Chronic Obstructive Pulmonary Disease (COPD), Neuromuscular Disease (NMD), and chest wall disorders.

Obstructive Sleep Apnea (OSA) is a form of Sleep Disordered Breathing (SDB), and is characterized by events including occlusion or obstruction of the upper air passage during sleep resulting from a combination of an abnormally small upper airway and the normal loss of muscle tone in the region of the tongue, soft palate and posterior oropharyngeal wall. Central Sleep Apnea (CSA) is another form of SDB that results when the brain temporarily stops sending signals to the muscles that control breathing. More generally, an apnea generally refers to the cessation of breathing caused by blockage of the air or the stopping of the breathing function. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

Cheyne-Stokes Respiration (CSR) is another form of SDB. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

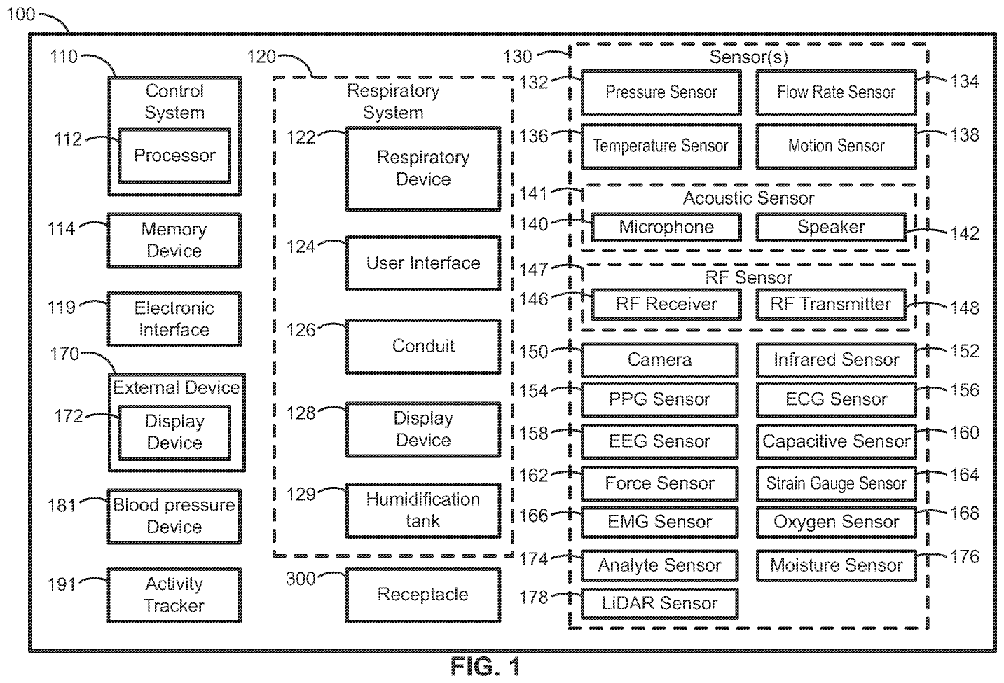

Referring to FIG. 1, a system 100, according to some implementations of the present disclosure, is illustrated. The system 100 is for providing a variety of different sensors related to a user's use of a respiratory system, among other uses. The system 100 includes a control system 110, a memory device 114, an electronic interface 119, one or more sensors 130, and one or more external devices 170. In some implementations, the system 100 further includes a respiratory system 120 that includes a respiratory device 122, a blood pressure device 181, an activity tracker 191, or any combination thereof. The system 100 can be used to deliver a substance to the user's airway.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 can be coupled to and/or positioned within, for example, a housing of the external device 170, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of any one or more of the sensors 130. Like the control system 110, the memory device 114 can be centralized (within one such housing) or decentralized (within two or more of such housings, which are physically distinct).

In some implementations, the memory device 114 (FIG. 1) stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a family medical history, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) test result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

The electronic interface 119 is configured to receive data (e.g., physiological and/or audio data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, an IR communication protocol, over a cellular network, over any other optical communication protocol, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the external device 170. In other implementations, the electronic interface 119 is coupled to or integrated (e.g., in a housing) with the control system 110 and/or the memory device 114.

As noted above, in some implementations, the system 100 optionally includes a respiratory system 120 (also referred to as a respiratory therapy system). The respiratory system 120 can include a respiratory device 122 (also referred to as a respiratory pressure therapy device), a user interface 124, a conduit 126 (also referred to as a tube or an air circuit), a display device 128, a humidification tank 129, or any combination thereof. The respiratory device 122, the user interface 124, and the conduit 126 form an air pathway of the respiratory system 120. In some implementations, the control system 110, the memory device 114, the display device 128, one or more of the sensors 130, and the humidification tank 129 are part of the respiratory device 122. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory system 120 is generally used to treat individuals suffering from one or more sleep-related respiratory disorders (e.g., obstructive sleep apnea, central sleep apnea, or mixed sleep apnea), other respiratory disorders such as COPD, or other disorders leading to respiratory insufficiency, that may manifest either during sleep or wakefulness.

The respiratory device 122 is generally used to generate pressurized air that is delivered to a user (e.g., using one or more motors that drive one or more compressors). In some implementations, the respiratory device 122 generates continuous constant air pressure that is delivered to the user. In other implementations, the respiratory device 122 generates two or more predetermined pressures (e.g., a first predetermined air pressure and a second predetermined air pressure). In still other implementations, the respiratory device 122 is configured to generate a variety of different air pressures within a predetermined range. For example, the respiratory device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory device 122 can also deliver pressurized air at a predetermined flow rate between, for example, about −20 L/min and about 150 L/min, while maintaining a positive pressure (relative to the ambient pressure). In some implementations, the control system 110, the memory device 114, the electronic interface 119, or any combination thereof can be coupled to and/or positioned within a housing of the respiratory device 122.

The user interface 124 engages a portion of the user's face and delivers pressurized air from the respiratory device 122 to the user's airway to aid in preventing the airway from narrowing and/or collapsing during sleep. This may also increase the user's oxygen intake during sleep. Depending upon the therapy to be applied, the user interface 124 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

The system also includes a receptacle 300 configured to receive and hold the substance. In the illustrated implementation, the receptacle 300 is shown as being separate from the respiratory system 120. In these implementations, the receptacle 300 can be a physical separate component that can be added to the respiratory system 120 when in use, for example by physical coupling the receptacle 300 to any component of the respiratory system 120. In other implementations, the receptacle 300 may be integrally built into any of the components of the respiratory system 120, for example being positioned inside the respiratory device 122, or as part of the user interface 124 or the conduit 126.

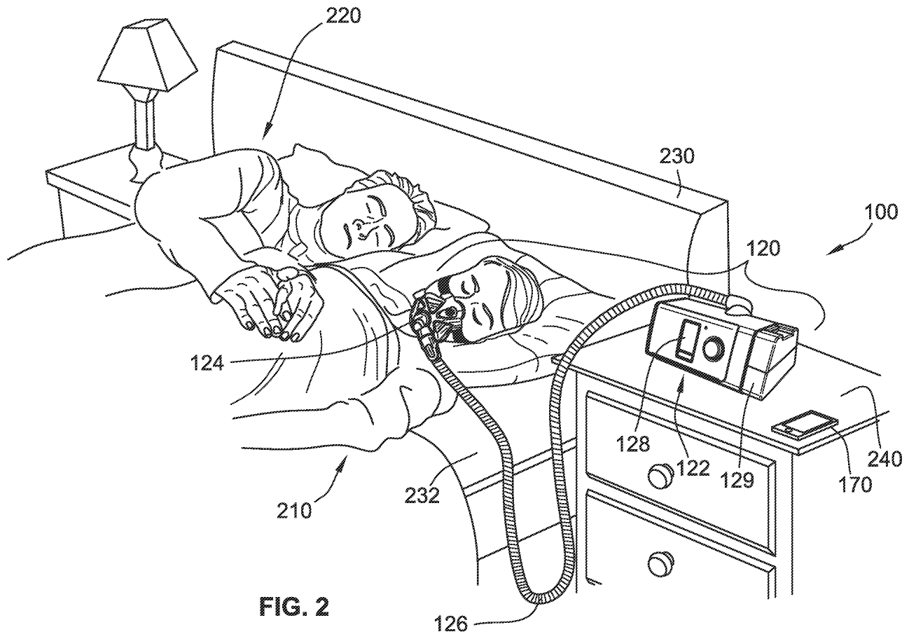

As shown in FIG. 2, in some implementations, the user interface 124 is or includes a facial mask that covers the nose and mouth of the user. Alternatively, the user interface 124 is or includes a nasal mask that provides air to the nose of the user or a nasal pillow mask that delivers air directly to the nostrils of the user. The user interface 124 can include a strap assembly that has a plurality of straps (e.g., including hook and loop fasteners) for positioning and/or stabilizing the user interface 124 on a portion of the user interface 124 on a desired location of the user (e.g., the face), and a conformal cushion (e.g., silicone, plastic, foam, etc.) that aids in providing an air-tight seal between the user interface 124 and the user. The user interface 124 can also include one or more vents for permitting the escape of carbon dioxide and other gases exhaled by the user 210. In other implementations, the user interface 124 includes a mouthpiece (e.g., a night guard mouthpiece molded to conform to the user's teeth, a mandibular repositioning device, etc.).

The conduit 126 allows the flow of air between two components of a respiratory system 120, such as the respiratory device 122 and the user interface 124. In some implementations, there can be separate limbs of the conduit for inhalation and exhalation. In other implementations, a single limb conduit is used for both inhalation and exhalation.

One or more of the respiratory device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be used, for example, to measure the air pressure and/or flow rate of pressurized air supplied by the respiratory device 122.

The display device 128 is generally used to display image(s) including still images, video images, or both and/or information regarding the respiratory device 122. For example, the display device 128 can provide information regarding the status of the respiratory device 122 (e.g., whether the respiratory device 122 is on/off, the pressure of the air being delivered by the respiratory device 122, the temperature of the air being delivered by the respiratory device 122, etc.) and/or other information (e.g., a sleep score or a therapy score (also referred to as a myAir™ score), the current date/time, personal information for the user 210, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory device 122.

The humidification tank 129 is coupled to or integrated in the respiratory device 122 and includes a reservoir of water that can be used to humidify the pressurized air delivered from the respiratory device 122. The respiratory device 122 can include a heater to heat the water in the humidification tank 129 in order to humidify the pressurized air provided to the user. Additionally, in some implementations, the conduit 126 can also include a heating element (e.g., coupled to and/or imbedded in the conduit 126) that heats the pressurized air delivered to the user. In other implementations, the respiratory device 122 or the conduit 126 can include a waterless humidifier. The waterless humidifier can incorporate sensors that interface with other sensor positioned elsewhere in system 100.

The respiratory system 120 can be used, for example, as a ventilator or a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based at least in part on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A user 210 of the respiratory system 120 and a bed partner 220 are located in a bed 230 and are laying on a mattress 232. The user interface 124 (e.g., a full facial mask) can be worn by the user 210 during a sleep session. The user interface 124 is fluidly coupled and/or connected to the respiratory device 122 via the conduit 126. In turn, the respiratory device 122 delivers pressurized air to the user 210 via the conduit 126 and the user interface 124 to increase the air pressure in the throat of the user 210 to aid in preventing the airway from closing and/or narrowing during sleep. The respiratory device 122 can be positioned on a nightstand 240 that is directly adjacent to the bed 230 as shown in FIG. 2, or more generally, on any surface or structure that is generally adjacent to the bed 230 and/or the user 210.

Referring to back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a radio-frequency (RF) transmitter 148, a camera 150, an infrared (IR) sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a light detection and ranging (LiDAR) sensor 178, or any combination thereof. Generally, each of the one or sensors 130 are configured to output sensor data that is received and stored in the memory device 114 or one or more other memory devices. The sensors 130 can also include, an electrooculography (EOG) sensor, a peripheral oxygen saturation (SpO₂) sensor, a galvanic skin response (GSR) sensor, a carbon dioxide (CO₂) sensor, or any combination thereof.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the IR sensor 152, the PPG sensor 154, the ECG sensor 156, the EEG sensor 158, the capacitive sensor 160, the force sensor 162, the strain gauge sensor 164, the EMG sensor 166, the oxygen sensor 168, the analyte sensor 174, the moisture sensor 176, and the LidAR sensor 178, more generally, the one or more sensors 130 can include any combination and any number of each of the sensors described and/or shown herein.

The one or more sensors 130 can be used to generate, for example physiological data, audio data, or both. Physiological data generated by one or more of the sensors 130 can be used by the control system 110 to determine a sleep-wake signal associated with a user during the sleep session and one or more sleep-related parameters. The sleep-wake signal can be indicative of one or more sleep stages, including sleep, wakefulness, relaxed wakefulness, micro-awakenings, or distinct sleep stages such as a rapid eye movement (REM) stage, a first non-REM stage (often referred to as “N1”), a second non-REM stage (often referred to as “N2”), a third non-REM stage (often referred to as “N3”), or any combination thereof.

The sleep-wake signal can also be timestamped to indicate a time that the user enters the bed, a time that the user exits the bed, a time that the user attempts to fall asleep, etc. The sleep-wake signal can be measured one or more of the sensors 130 during the sleep session at a predetermined sampling rate, such as, for example, one sample per second, one sample per 30 seconds, one sample per minute, etc. Examples of the one or more sleep-related parameters that can be determined for the user during the sleep session based at least in part on the sleep-wake signal include a total time in bed, a total sleep time, a total wake time, a sleep onset latency, a wake-after-sleep-onset parameter, a sleep efficiency, a fragmentation index, an amount of time to fall asleep, a consistency of breathing rate, a fall asleep time, a wake time, a rate of sleep disturbances, a number of movements, or any combination thereof.

Physiological data and/or audio data generated by the one or more sensors 130 can also be used to determine a respiration signal associated with a user during a sleep session. the respiration signal is generally indicative of respiration or breathing of the user during the sleep session. The respiration signal can be indicative of, for example, a respiration rate, a respiration rate variability, an inspiration amplitude, an expiration amplitude, an inspiration-expiration amplitude ratio, an inspiration-expiration duration ratio, a number of events per hour, a pattern of events, pressure settings of the respiratory device 122, or any combination thereof. The event(s) can include snoring, apneas, central apneas, obstructive apneas, mixed apneas, hypopneas, a mask leak (e.g., from the user interface 124), a restless leg, a sleeping disorder, choking, an increased heart rate, a heart rate variation, labored breathing, an asthma attack, an epileptic episode, a seizure, a fever, a cough, a sneeze, a snore, a gasp, the presence of an illness such as the common cold or the flu, an elevated stress level, etc.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the user of the respiratory system 120 and/or ambient pressure. In such implementations, the pressure sensor 132 can be coupled to or integrated in the respiratory device 122. The pressure sensor 132 can be, for example, a capacitive sensor, an electromagnetic sensor, an inductive sensor, a resistive sensor, a piezoelectric sensor, a strain-gauge sensor, an optical sensor, a potentiometric sensor, or any combination thereof. In one example, the pressure sensor 132 can be used to determine a blood pressure of the user.

The flow rate sensor 134 outputs flow rate data that can be stored in the memory device 114 and/or analyzed by the processor 112 of the control system 110. In some implementations, the flow rate sensor 134 is used to determine an air flow rate from the respiratory device 122, an air flow rate through the conduit 126, an air flow rate through the user interface 124, or any combination thereof. In such implementations, the flow rate sensor 134 can be coupled to or integrated in the respiratory device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The temperature sensor 136 outputs temperature data that can be stored in the memory device 114 and/or analyzed by the processor 112 of the control system 110. In some implementations, the temperature sensor 136 generates temperatures data indicative of a core body temperature of the user 210 (FIG. 2), a skin temperature of the user 210, a temperature of the air flowing from the respiratory device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user 210 during the sleep session, and/or detect movement of any of the components of the respiratory system 120, such as the respiratory device 122, the user interface 124, or the conduit 126. The motion sensor 138 can include one or more inertial sensors, such as accelerometers, gyroscopes, and magnetometers. The motion sensor 138 can be used to detect motion or acceleration associated with arterial pulses, such as pulses in or around the face of the user 210 and proximal to the user interface 124, and configured to detect features of the pulse shape, speed, amplitude, or volume.

The microphone 140 outputs sound data that can be stored in the memory device 114 and/or analyzed by the processor 112 of the control system 110. The audio data generated by the microphone 140 is reproducible as one or more sound(s) during a sleep session (e.g., sounds from the user 210) to determine (e.g., using the control system 110) one or more sleep-related parameters, as described in further detail herein. The audio data from the microphone 140 can also be used to identifying (e.g., using the control system 110) an event experienced by the user during the sleep session, as described in further detail herein. The microphone 140 can be coupled to or integrated in the respiratory device 122, the user interface 124, the conduit 126, or the external device 170.

The speaker 142 outputs sound waves that are audible to a user of the system 100 (e.g., the user 210 of FIG. 2). The speaker 142 can be used, for example, as an alarm clock or to play an alert or message to the user 210 (e.g., in response to an event). In some implementations, the speaker 142 can be used to communicate the audio data generated by the microphone 140 to the user. The speaker 142 can be coupled to or integrated in the respiratory device 122, the user interface 124, the conduit 126, or the external device 170.

The microphone 140 and the speaker 142 can be used as separate devices. In some implementations, the microphone 140 and the speaker 142 can be combined into an acoustic sensor 141, as described in, for example, WO 2018/050913, which is hereby incorporated by reference herein in its entirety. In such implementations, the speaker 142 generates or emits sound waves at a predetermined interval and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the user 210 or the bed partner 220 (FIG. 2). Based at least in part on the data from the microphone 140 and/or the speaker 142, the control system 110 can determine a location of the user 210 (FIG. 2) and/or one or more of the sleep-related parameters described in herein. In some implementations, the speaker 142 is a bone conduction speaker. In some implementations, the one or more sensors 130 include (i) a first microphone that is the same or similar to the microphone 140, and is integrated into the acoustic sensor 141 and (ii) a second microphone that is the same as or similar to the microphone 140, but is separate and distinct from the first microphone that is integrated into the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of the user 210 (FIG. 2) and/or one or more of the sleep-related parameters described herein. An RF receiver (either the RF receiver 146 and the RF transmitter 148 or another RF pair) can also be used for wireless communication between the control system 110, the respiratory device 122, the one or more sensors 130, the external device 170, or any combination thereof. While the RF receiver 146 and RF transmitter 148 are shown as being separate and distinct elements in FIG. 1, in some implementations, the RF receiver 146 and RF transmitter 148 are combined as a part of a radio-frequency (RF) sensor 147. In some such implementations, the RF sensor 147 includes a control circuit. The specific format of the RF communication could be WiFi, Bluetooth, etc.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor 147. The WiFi router and satellites continuously communicate with one another using WiFi signals. The WiFi mesh system can be used to generate motion data based at least in part on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or a combination thereof) that can be stored in the memory device 114. The image data from the camera 150 can be used by the control system 110 to determine one or more of the sleep-related parameters described herein. For example, the image data from the camera 150 can be used to identify a location of the user, to determine a time when the user 210 enters the bed 230 (FIG. 2), and to determine a time when the user 210 exits the bed 230. The camera 150 can also be used to track eye movements, pupil dilation (if one or both of the user 210's eyes are open), blink rate, or any changes during REM sleep. The camera 150 can also be used to track the position of the user, which can impact the duration and/or severity of apneic episodes in users with positional obstructive sleep apnea.

The IR sensor 152 outputs infrared image data reproducible as one or more infrared images (e.g., still images, video images, or both) that can be stored in the memory device 114. The infrared data from the IR sensor 152 can be used to determine one or more sleep-related parameters during the sleep session, including a temperature of the user 210 and/or movement of the user 210. The IR sensor 152 can also be used in conjunction with the camera 150 when measuring the presence, location, and/or movement of the user 210. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the user 210 (FIG. 2) that can be used to determine one or more sleep-related parameters, such as, for example, a heart rate, a heart rate pattern, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the user 210, embedded in clothing and/or fabric that is worn by the user 210, embedded in and/or coupled to the user interface 124 and/or its associated headgear (e.g., straps, etc.), etc.

The ECG sensor 156 outputs physiological data associated with electrical activity of the heart of the user 210. In some implementations, the ECG sensor 156 includes one or more electrodes that are positioned on or around a portion of the user 210 during the sleep session. The physiological data from the ECG sensor 156 can be used, for example, to determine one or more of the sleep-related parameters described herein.

The EEG sensor 158 outputs physiological data associated with electrical activity of the brain of the user 210. In some implementations, the EEG sensor 158 includes one or more electrodes that are positioned on or around the scalp of the user 210 during the sleep session. The physiological data from the EEG sensor 158 can be used, for example, to determine a sleep stage of the user 210 at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the user 210. The data output by the analyte sensor 174 can be stored in the memory device 114 and used by the control system 110 to determine the identity and concentration of any analytes in the user 210's breath. In some implementations, the analyte sensor 174 is positioned near a mouth of the user 210 to detect analytes in breath exhaled from the user 210's mouth. For example, when the user interface 124 is a facial mask that covers the nose and mouth of the user 210, the analyte sensor 174 can be positioned within the facial mask to monitor the user 210's mouth breathing. In other implementations, such as when the user interface 124 is a nasal mask or a nasal pillow mask, the analyte sensor 174 can be positioned near the nose of the user 210 to detect analytes in breath exhaled through the user 210's nose. In still other implementations, the analyte sensor 174 can be positioned near the user 210's mouth when the user interface 124 is a nasal mask or a nasal pillow mask. In this implementation, the analyte sensor 174 can be used to detect whether any air is inadvertently leaking from the user 210's mouth. In some implementations, the analyte sensor 174 is a volatile organic compound (VOC) sensor that can be used to detect carbon-based chemicals or compounds, such as carbon dioxide. In some implementations, the analyte sensor 174 can also be used to detect whether the user 210 is breathing through their nose or mouth. For example, if the data output by an analyte sensor 174 positioned near the mouth of the user 210 or within the facial mask (in implementations where the user interface 124 is a facial mask) detects the presence of an analyte, the control system 110 can use this data as an indication that the user 210 is breathing through their mouth.

The moisture sensor 176 outputs data that can be stored in the memory device 114 and used by the control system 110. The moisture sensor 176 can be used to detect moisture in various areas surrounding the user (e.g., inside the conduit 126 or the user interface 124, near the user 210's face, near the connection between the conduit 126 and the user interface 124, near the connection between the conduit 126 and the respiratory device 122, etc.). Thus, in some implementations, the moisture sensor 176 can be coupled to or integrated into the user interface 124 or in the conduit 126 to monitor the humidity of the pressurized air from the respiratory device 122. In other implementations, the moisture sensor 176 is placed near any area where moisture levels need to be monitored. The moisture sensor 176 can also be used to monitor the humidity of the ambient environment surrounding the user 210, for example the air inside the user 210's bedroom. The moisture sensor 176 can also be used to track the user 210's biometric response to environmental changes.

One or more LiDAR sensors 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 178 can measure and map an area extending 5 meters or more away from the sensor. The LiDAR data can be fused with point cloud data estimated by an electromagnetic RADAR sensor, for example. The LiDAR sensor 178 may also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

While shown separately in FIG. 1, any combination of the one or more sensors 130 can be integrated in and/or coupled to any one or more of the components of the system 100, including the respiratory device 122, the user interface 124, the conduit 126, the humidification tank 129, the control system 110, the external device 170, or any combination thereof. For example, the acoustic sensor 141 and/or the RF sensor 147 can be integrated in and/or coupled to the external device 170. In such implementations, the external device 170 can be considered a secondary device that generates additional or secondary data for use by the system 100 (e.g., the control system 110) according to some aspects of the present disclosure. In some implementations, the pressure sensor 132 and/or the flow rate sensor 134 are integrated into and/or coupled to the respiratory device 122. In some implementations, at least one of the one or more sensors 130 is not coupled to the respiratory device 122, the control system 110, or the external device 170, and is positioned generally adjacent to the user 210 during the sleep session (e.g., positioned on or in contact with a portion of the user 210, worn by the user 210, coupled to or positioned on the nightstand, coupled to the mattress, coupled to the ceiling, etc.). More generally, the one or more sensors 130 can be positioned at any suitable location relative to the user 210 such that the one or more sensors 130 can generate physiological data associated with the user 210 and/or the bed partner 220 during one or more sleep session.

The data from the one or more sensors 130 can be analyzed to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, an average duration of events, a range of event durations, a ratio between the number of different events, a sleep stage, an apnea-hypopnea index (AHI), or any combination thereof. The one or more events can include snoring, apneas, central apneas, obstructive apneas, mixed apneas, hypopneas, an intentional user interface leak, an unintentional user interface leak, a mouth leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 130, or from other types of data.

The external device 170 (FIG. 1) includes a display device 172. The external device 170 can be, for example, a mobile device such as a smart phone, a tablet, a laptop, or the like. Alternatively, the external device 170 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human user interacting with the external device 170. In some implementations, one or more user devices can be used by and/or included in the system 100.

The blood pressure device 181 is generally used to aid in generating physiological data for determining one or more blood pressure measurements associated with a user. The blood pressure device 181 can include at least one of the one or more sensors 130 to measure, for example, a systolic blood pressure component and/or a diastolic blood pressure component.

In some implementations, the blood pressure device 181 is a sphygmomanometer including an inflatable cuff that can be worn by a user and a pressure sensor (e.g., the pressure sensor 132 described herein). For example, as shown in the example of FIG. 2, the blood pressure device 181 can be worn on an upper arm of the user 210. In such implementations where the blood pressure device 181 is a sphygmomanometer, the blood pressure device 181 also includes a pump (e.g., a manually operated bulb) for inflating the cuff. In some implementations, the blood pressure device 181 is coupled to the respiratory device 122 of the respiratory system 120, which in turn delivers pressurized air to inflate the cuff. More generally, the blood pressure device 181 can be communicatively coupled with, and/or physically integrated in (e.g., within a housing), the control system 110, the memory device 114, the respiratory system 120, the external device 170, and/or the activity tracker 191.

The activity tracker 191 is generally used to aid in generating physiological data for determining an activity measurement associated with the user. The activity measurement can include, for example, a number of steps, a distance traveled, a number of steps climbed, a duration of physical activity, a type of physical activity, an intensity of physical activity, time spent standing, a respiration rate, an average respiration rate, a resting respiration rate, a maximum respiration rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. The activity tracker 191 includes one or more of the sensors 130 described herein, such as, for example, the motion sensor 138 (e.g., one or more accelerometers and/or gyroscopes), the PPG sensor 154, and/or the ECG sensor 156.

In some implementations, the activity tracker 191 is a wearable device that can be worn by the user, such as a smartwatch, a wristband, a ring, or a patch. For example, referring to FIG. 2, the activity tracker 191 is worn on a wrist of the user 210. The activity tracker 191 can also be coupled to or integrated a garment or clothing that is worn by the user. Alternatively, still, the activity tracker 191 can also be coupled to or integrated in (e.g., within the same housing) the external device 170. More generally, the activity tracker 191 can be communicatively coupled with, or physically integrated in (e.g., within a housing), the control system 110, the memory device 114, the respiratory system 120, the external device 170, and/or the blood pressure device 181.

While the control system 110 and the memory device 114 are described and shown in FIG. 1 as being a separate and distinct component of the system 100, in some implementations, the control system 110 and/or the memory device 114 are integrated in the external device 170 and/or the respiratory device 122. Alternatively, in some implementations, the control system 110 or a portion thereof (e.g., the processor 112) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system for canceling noises during use of the respiratory system 120, according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, and the external device 170. As yet another example, a third alternative system includes the control system 110, the memory device 114, the respiratory system 120, at least one of the one or more sensors 130, and the external device 170. As a further example, a fourth alternative system includes the control system 110, the memory device 114, the respiratory system 120, at least one of the one or more sensors 130, the external device 170, and the blood pressure device 181 and/or activity tracker 191. Thus, various systems for delivering a substance into the user's airway can be formed using any portion or portions of the components shown and described herein and/or in combination with one or more other components.

As used herein, a sleep session can be defined in a number of ways based at least in part on, for example, an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 172 of the external device 170 (FIG. 1) to manually initiate or terminate the sleep session.

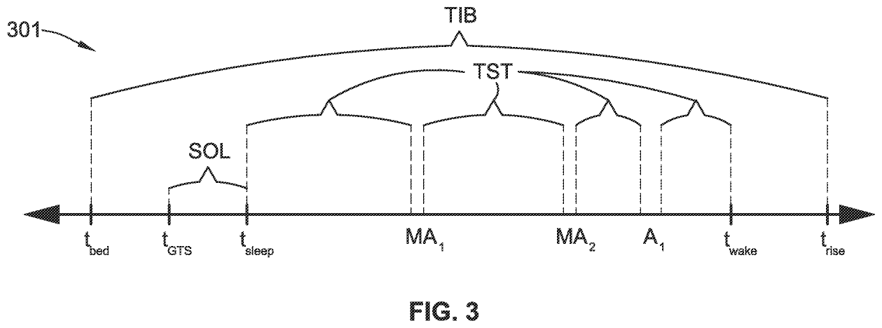

Referring to FIG. 3, an exemplary timeline 301 for a sleep session is illustrated. The timeline 301 includes an enter bed time (t_{bed}), a go-to-sleep time (t_{GTS}), an initial sleep time (tₛₗₑₑₚ), a first micro-awakening MA₁, a second micro-awakening MA₂, an awakening A, a wake-up time (t_{wake}), and a rising time (tᵣᵢₛₑ).

The enter bed time t_{bed} is associated with the time that the user initially enters the bed (e.g., bed 230 in FIG. 2) prior to falling asleep (e.g., when the user lies down or sits in the bed). The enter bed time t_{bed} can be identified based at least in part on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the external device 170, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based at least in part on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based at least in part on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based at least in part on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based at least in part on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based at least in part on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 301 of FIG. 3, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

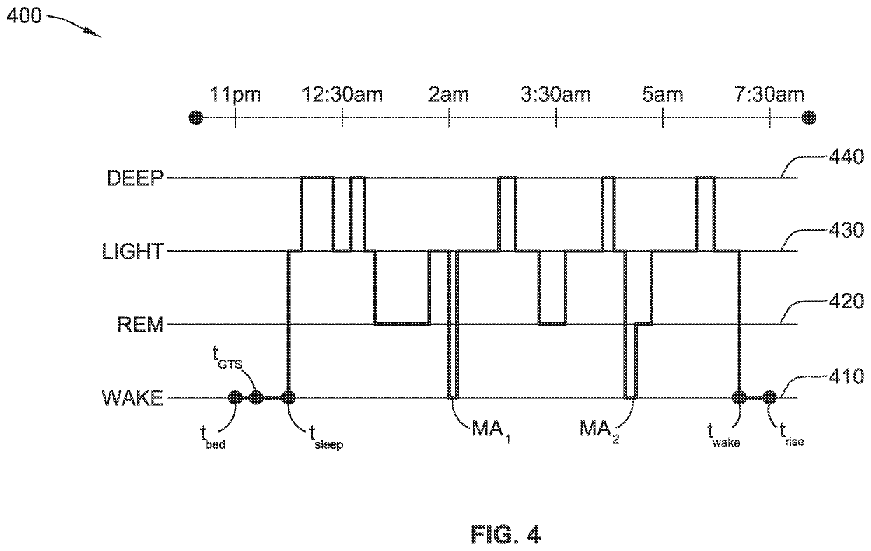

Referring to FIG. 4, an exemplary hypnogram 400 corresponding to the timeline 301 (FIG. 3), according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410-440 is indicative of the sleep stage at any given time during the sleep session.

The sleep-wake signal 401 can be generated based at least in part on physiological data associated with the user (e.g., generated by one or more of the sensors 130 described herein). The sleep-wake signal can be indicative of one or more sleep stages, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration amplitude ratio, an inspiration-expiration duration ratio, a number of events per hour, a pattern of events, or any combination thereof. Information describing the sleep-wake signal can be stored in the memory device 114.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based at least in part on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 130 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based at least in part on, for example, data generated by the motion sensor 138, the microphone 140, the camera 150, or any combination thereof. The go-to-sleep time can be determined based at least in part on, for example, data from the motion sensor 138 (e.g., data indicative of no movement by the user), data from the camera 150 (e.g., data indicative of no movement by the user and/or that the user has turned off the lights), data from the microphone 140 (e.g., data indicative of the using turning off a TV), data from the external device 170 (e.g., data indicative of the user no longer using the external device 170), data from the pressure sensor 132 and/or the flow rate sensor 134 (e.g., data indicative of the user turning on the respiratory device 122, data indicative of the user donning the user interface 124, etc.), or any combination thereof.

Continuous positive airway pressure (CPAP) systems are often used to treat individuals suffering from sleep-related respiratory disorders. Generally, the user of a CPAP system wears a user interface (such as a mask), which delivers pressurized air from a respiratory device into the throat of the user to aid in preventing the airway from narrowing and/or collapsing during sleep, thereby increasing the user's oxygen intake. Many CPAP systems generate audible noise during use that can interfere with or interrupt the user's sleep. This noise often arises from the operation of a motor within the respiratory device that generates the pressurized air. Further, noise can arise from air leaks in CPAP systems (e.g., from a mask of the CPAP system). Detecting and canceling such noises during operation of the CPAP system is useful in aiding users and their bed partners to have high quality sleep that is not interrupted by such noises.

System 100 can be used to deliver at least a portion of a substance from the receptacle 300 to the air pathway the user based at least in part on the physiological data, the sleep-related parameters, other data or information, or any combination thereof. Generally, modifying the delivery of the portion of the substance into the air pathway can include (i) initiating the delivery of the substance into the air pathway, (ii) ending the delivery of the portion of the substance into the air pathway, (iii) modifying an amount of the substance delivered into the air pathway, (iv) modifying a temporal characteristic of the delivery of the portion of the substance into the air pathway, (v) modifying a quantitative characteristic of the delivery of the portion of the substance into the air pathway, (vi) modifying any parameter associated with the delivery of the substance into the air pathway, or (vii) any combination of (i)-(vi). Modifying the temporal characteristic of the delivery of the portion of the substance into the air pathway can include changing the rate at which the substance is delivered, starting and/or finishing at different times, continuing for different time periods, changing the time distribution or characteristics of the delivery, changing the amount distribution independently of the time distribution, etc. The independent time and amount variation ensures that, apart from varying the frequency of the release of the substance, one can vary the amount of substance released each time. In this manner, a number of different combination of release frequencies and release amounts (e.g., higher frequency but lower release amount, higher frequency and higher amount, lower frequency and higher amount, lower frequency and lower amount, etc.) can be achieved. Other modifications to the delivery of the portion of the substance into the air pathway can also be utilized.

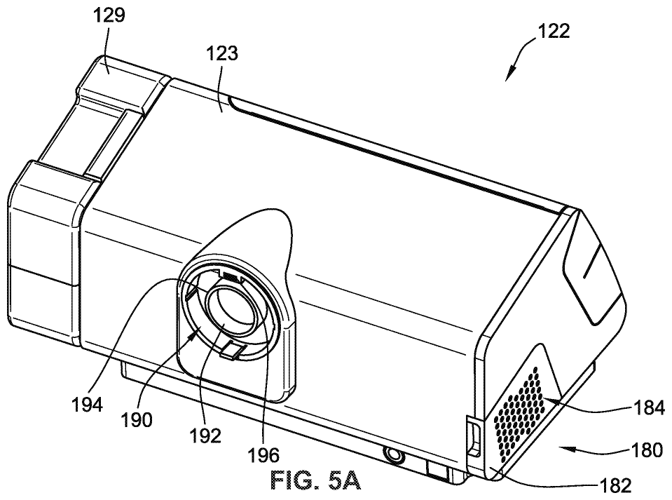

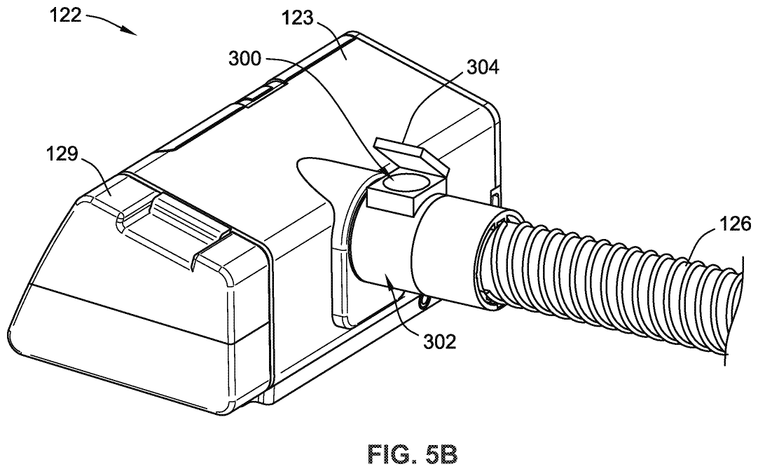

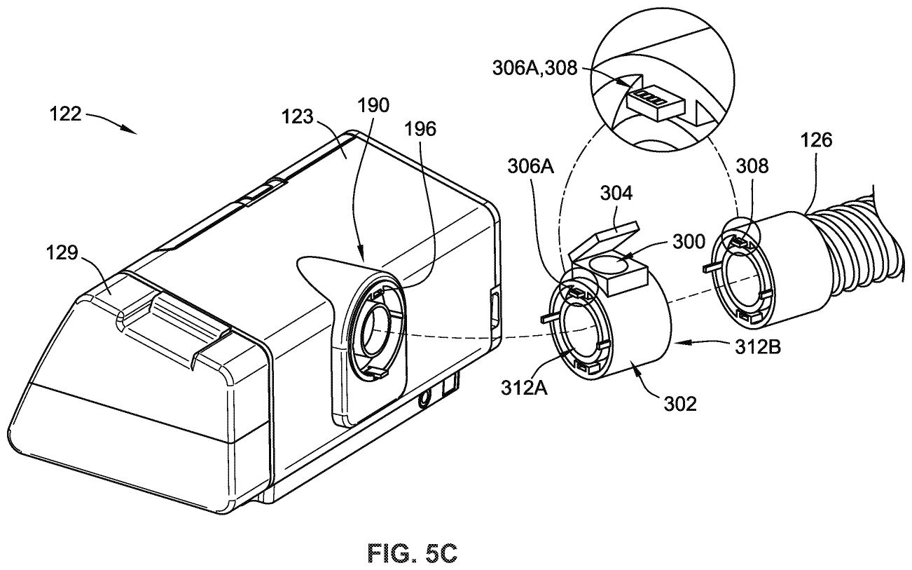

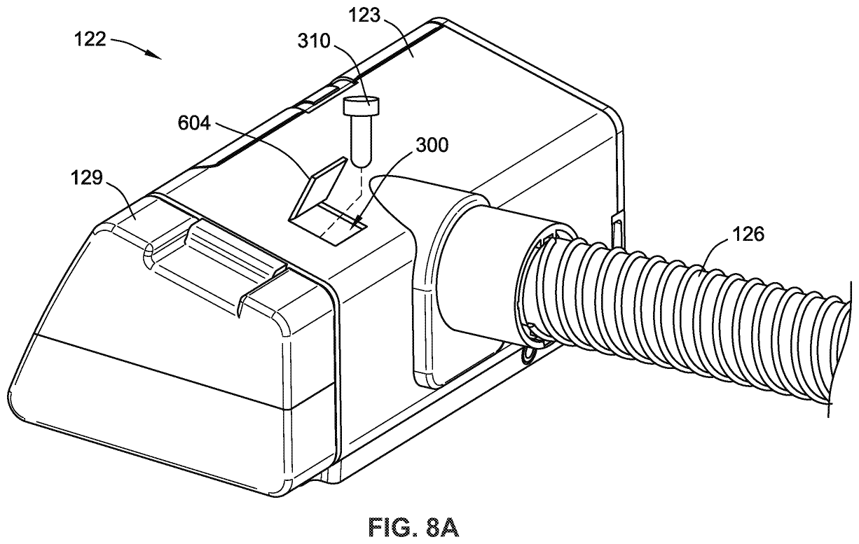

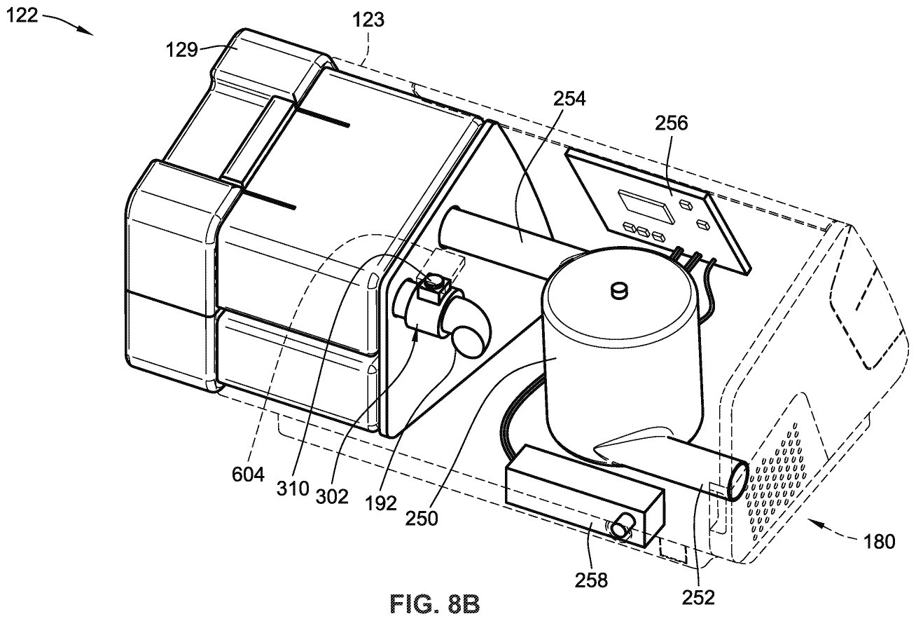

FIG. 5A shows a perspective view of the back side of the respiratory device 122 that includes a housing 123, an air inlet 180, and an air outlet 190. The air inlet 180 includes an inlet cover 182 movable between a closed position and an open position. The air inlet cover 182 includes one or more air inlet apertures 184 defined therein. The respiratory device 122 includes a blower motor 250 (see FIG. 8B) configured to draw air in through the one or more air inlet apertures 184 defined in the air inlet cover 182. The motor is further configured to cause pressurized air to flow through the humidification tank 129 and out of the air outlet 190. The conduit 126 can be fluidly coupled to the air outlet 190, such that the air flows from the air outlet 190 and into the conduit 126. The air outlet 190 is partially formed by an internal conduit 192 extending through the housing 123 from the interior of the respiratory device 122. A seal 194 is positioned around the end of the internal conduit 192 to ensure that substantially all of the air that exits through the air outlet 190 flows into the conduit 126.

FIGS. 5B and 5C show a perspective view of an implementation of the receptacle 300 where the receptacle 300 is formed as part of an injection plug 302, and the respiratory device 122 is configured to engage the receptacle 300 by coupling with the injection plug 302. The injection plug 302 is used to deliver a substance into the air pathway of the respiratory system 120 so that the substance reaches the airway of the user 210. As is discussed in more detail herein, the substance can be a medicament, such as anti-inflammatory medicine, medicine to treat an asthma attack, medicine to treat a heart attack, etc. Generally, any type of medicament that is used to treat any ailment, symptom, disease, etc. can be delivered to the airway of the user 210 using the injection plug 302. When the substance is a medicament, the substance generally includes one or more active ingredients, and one or more excipients. The excipients serve as the medium for conveying the active ingredient, and can include substances such as bulking agents, fillers, diluents, antiadherents, binders, coatings, colors, disintegrants, flavors, glidants, lubricants, preservatives, sorbents, sweeteners, vehicles, or any combinations thereof. The active ingredient is generally the portion of the medicament that actually causes the effect brought on by the medicament.

The substance could also be an aroma compound (e.g., a substance that delivers scents and/or aromas to the airway of the user 210), a sleep-aid (e.g., a substance that aids the user 210 in falling asleep), a consciousness-arousing compound (e.g., a substance that aids the user 210 in waking up, also referred to as a sleep inhibitor), a cannabidiol oil, an essential oil (such as lavender, valerian, clary sage, sweet marjoram, roman chamomile, bergamot, etc.). The substance can generally be a solid, a liquid, a gas, or any combination thereof. The substance can alternatively or additionally include one or more nanoparticles.

In some implementations, the substance is configured to aid in (i) opening the airway of the user 210, (ii) managing (e.g., reducing, maintaining, or raising) a blood pressure of the user 210, (iii) managing (e.g., reducing, maintaining, or raising) a heart rate of the user 210, (iv) reducing a heart rate variability of the user 210, (v) stabilizing a heart rate of the user 210, (vi) managing (e.g., reducing, maintaining, or raising) an analyte (such as glucose) concentration in blood of the user 210, (vii) reducing inflammation in one or more portions of the user 210, (viii) managing (e.g., inducing or maintaining) sleep of the user 210, (ix) waking up the user 210, (x) reducing pain in one or more body parts of the user 210, (xi) improving a perception of wellbeing of the user 210 (e.g., making the user feel better by using aromatherapy), or (xii) any combination thereof. In some implementations, if the user has insomnia and has trouble sleeping, the substance is configured aid the user in falling asleep and staying asleep. Generally, the injection plug 302 can be used to deliver any type of substance into the air pathway of the respiratory system 120, such that the substance is delivered to the airway of the use along with the pressurized air.

As shown in FIGS. 5B and 5C, the injection plug 302 is positioned exterior to the respiratory device 122, and can be placed in-line with the air outlet 190 of the respiratory device 122 and the conduit 126. In this arrangement, the injection plug 302 is fluidly coupled between the air outlet 190 and the conduit 126, such that the injection plug 302 is in fluid communication with the air pathway. As the pressurized air is caused to exit the air outlet 190, the pressurized air flows through the injection plug 302 and into the conduit 126. In other implementations however, the injection plug 302 can have different arrangements. For example, the injection plug 302 could be fluidly coupled between the conduit 126 and the user interface 124. The injection plug 302 could also be fluidly coupled to the air inlet 180 of the respiratory device 122. In this implementation, the injection plug 302 can be considered to be fluidly coupled between the air inlet 180 of the respiratory device 122 and the atmosphere outside of the respiratory device 122. Thus, the injection plug 302 in FIGS. 5B and 5C is generally placed in-line with the air pathway such that the pressurized air flows through the injection plug 302 and the receptacle 300 when the pressurized air is directed to the airway of the user 210.

The injection plug 302 defines a receptacle 300 that is configured to receive and contain the substance therein. In the illustrated embodiment, the receptacle 300 is an open space that extends from the interior of the injection plug 302 to the periphery of the injection plug 302. The injection plug 302 includes a movable cover 304 that controls access to the receptacle 300. FIGS. 3B and 3C show the cover 304 in a first position. In the first position, the cover 304 is open, such that the substance can be inserted into the receptacle 300. In a second position, the cover 304 is closed, such that the substance cannot be inserted into the receptacle 300. The cover 304 is generally illustrated as being configured to pivot between the first position and the second position, for example via one or more hinges or one or more pivots. However, the cover 304 can be configured to be movable using any number of mechanisms or techniques. For example, the cover 304 could move in a single plane either horizontally or vertically between the first position and the second position. The cover 304 could also be configured to be twistable, such that rotation of the cover 304 moves the cover 304 between the first position and the second position. In still other implementations, the cover 304 could be attached to the injection plug 302 via a friction fit, a press fit, a snap fit, etc. In these implementations, opening the cover 304 to allow access to the receptacle 300 can include detaching the cover 304 from the injection plug 302. Closing the cover 304 to prevent access to the receptacle 300 can include attaching the cover 304 to the injection plug 302. The cover 304 can be manually movable by the user 210, can be controlled by the control system 110, or both.

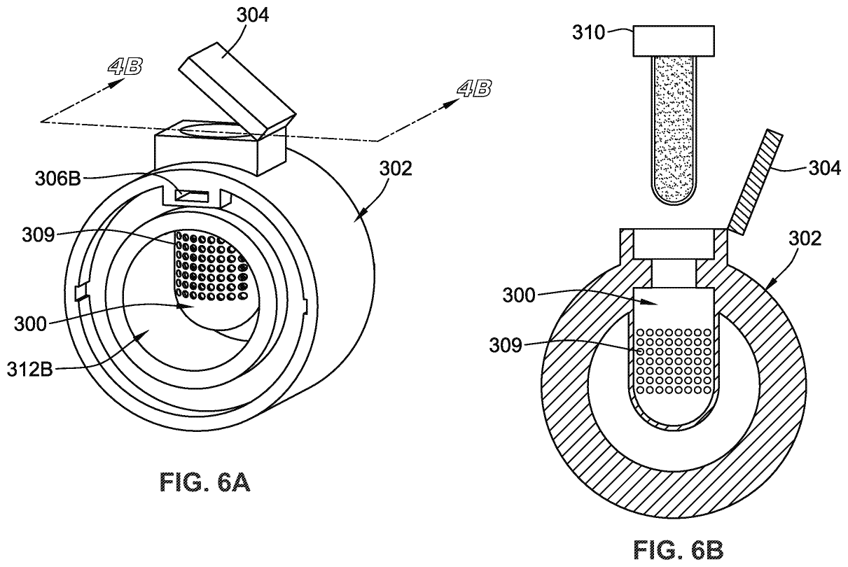

The injection plug 302 may arranged to contain, and facilitate the injection of, the substance included in the receptacle 300. While in some cases the injection plug 302 may include a controller for managing the functions and features of the injection plug 302, in other cases the injection plug 302 may need to receive control signals from the control system 110. When the injection plug 302 is disposed between the respiratory device 122 and the conduit 126, the injection plug 302 may also need to transfer electrical signals (e.g., sensing signals) coming from the user interface 124 to the respiratory device 122. To be able to effect all these functionalities, depending on its location, the injection plug 302 may need to be electrically connected to one or both of the respiratory device 122 and the user interface 124, as well as mechanically. Thus, the injection plug 302 can include one or more sets of electrical contacts that electrically couple the injection plug 302 to the respiratory device 122 and the conduit 126. A first set of electrical contacts 306A is illustrated in FIG. 5C. The first set of electrical contacts 306A generally protrudes from the injection plug 302, and is configured to mate with corresponding electrical contacts 196 recessed in the air outlet 190 of the respiratory device 122. On the opposing side of the injection plug 302 where the injection plug 302 couples with the conduit 126, the injection plug 302 can have a second set of electrical contacts 306B recessed in the injection plug 302 (best shown in FIG. 6A). The second set of electrical contacts 306B of the injection plug 302 can electrically couple with a corresponding set of electrical contacts 308 on the conduit 126.

The electrical contacts 196, 306A, 306B, 308 on the respiratory device 122, the injection plug 302, and the conduit 126 can be used in a variety of different manners. In some implementations, one or more of the electrical contacts 196, 306A, 306B, 308 are used to heat the air flowing through the injection plug 302 and the conduit 126, either directly or by heating a separate heating element. One or more of the electrical contacts 196, 306A, 306B, 308 can also act as a heater that is configured to heat the substance and cause some or all of the substance to evaporate, again either directly or by heating a separate heating element. The evaporation of the substance can be selectively controlled manually by the user 210, and/or by the control system 110.

In additional or alternative implementations, one or more of the electrical contacts 196, 306A, 306B, 308 are used to electrically couple the injection plug 302 and/or the conduit 126 to the control system 110. In these implementations, the control system 110 can control the injection plug 302 so as to selectively release the substance into the air pathway of the respiratory system 120. As is discussed in more detail herein, the control system 110 can control the release of the substance into the air pathway based at least in part on a variety of different factors, including physiological data related to the user 210 and the sleep session. The control system 110 can also be used to control the movement of the cover 304 between the first (open) position and the second (closed) position.

FIGS. 6A and 6B show a first implementation of the injection plug 302. In this implementation, air flowing through the injection plug 302 causes the substance to be delivered into the air pathway. The receptacle 300 and the injection plug 302 have an inlet 312A (best shown in FIG. 5C) and an outlet 312B. In the implementation shown in FIGS. 6A and 6B, the inlet 312A is formed generally from the open front end of the injection plug 302 and the apertures 309 defined in the front end of the receptacle 300, while the outlet 312B is formed generally from the open back end of the injection plug 302 and the apertures 309 defined in the back end of the receptacle 300. However, the inlet 312A and the outlet 312B could be formed in any suitable fashion.

The apertures 309 are defined in the receptacle 300 where the substance is contained during use. In this implementation, both the inlet 312A and the outlet 312B are in direct or indirect fluid communication with the air pathway. The interior of the receptacle 300 where the substance is contained is also in direct or indirect fluid communication with the air pathway due to the presence of the apertures in the receptacle 300. When the respiratory device 122 is in use, the pressurized air enters the injection plug 302 via the inlet 312A, passes through the receptacle 300 via the apertures, and exits the injection plug 302 via the outlet 312B. The pressurized air carries the substance out of the injection plug 302 and the receptacle 300 as the pressurized air flows through the injection plug 302 and the receptacle 300. Thus, the injection plug 302 can act as a nebulizer that aerosolizes the substance.

In some implementations where the injection plug 302 is located between the respiratory device 122 and the conduit 126, the inlet 312A is in fluid communication with the air outlet 190 of the respiratory device 122, and the outlet 312B is in fluid communication with the conduit 126. This implementation is illustrated in FIGS. 5B and 5C. In other implementations where the injection plug 302 is located between the conduit 126 and the user interface 124, the inlet 312A is in fluid communication with the conduit 126, and the outlet 312B is in fluid communication with the user interface 124. In still other implementations where the injection plug is located adjacent to the air inlet 180 of the respiratory device 122, the inlet 312A is in fluid communication with the atmosphere outside of the respiratory device 122, and the outlet 312B is in fluid communication with the air inlet 180 of the respiratory device 122. In still other implementations, the injection plug 302 could be located within the user interface 124 itself. In this implementation, the inlet 312A is in fluid communication with the user interface 124, and the outlet 312B is in fluid communication with the sealed environment between the user interface 124 and the mouth of the user 210. Thus, in some implementations, the substance could be delivered into the air pathway at or near the user interface 124 itself. In still further implementations, the injection plug 302 is located within the housing of the respiratory device 122 itself. In this implementations, the outlet 312B is in fluid communication with an interior of the air outlet 190 of the respiratory device 122, and the exterior of the air outlet 190 of the respiratory device 122 is in fluid communication the conduit 126. In other implementations, the injection plug 302 is located within the conduit 126. In these implementations, both the inlet 312A and the outlet 312B are in fluid communication with the conduit 126.

As shown in FIGS. 6A and 6B, the substance can be contained within a separate pod 310 (e.g., a sachet) that is itself received by the receptacle 300, such that the pod 310 is disposed within the injection plug 302. In these implementations, the pod 310 must be able to release the substance into the receptacle 300 in order for the pressurized air to carry the substance along the air pathway. For example, the interior of the receptacle 300 could contain a mechanism that pierces or otherwise opens the pod 310 when the pod 310 is initially inserted into the receptacle 300. The term pod is used herein to broadly describe any container containing the substance. The specific material and structure of the container will depend on the nature of the substance, which can be in liquid, solid, or gaseous form.

In some implementations, the injection plug 302 of FIGS. 6A and 6B includes a bypass valve fluidly coupled to the injection plug 302 and the receptacle 300. The bypass valve can be used to selectively alter the path of the pressurized air through the injection plug 302. For example, the bypass valve can have and/or form a first pathway that bypasses the receptacle 300, and a second pathway that is in fluid communication with the inlet 312A. In this example, the first pathway can be pass outside of the entire injection plug 302, or can pass through the interior of the injection plug 302 but not through the receptacle 300 (e.g., pass underneath the receptacle 300 with reference to FIGS. 6A and 6B). The second pathway passes through the receptacle 300 via the apertures defined by the receptacle 300. Generally, the bypass valve includes a mechanical element that diverts the pressurized air into either the first pathway or the second pathway.

The bypass valve can be used to direct all of the pressurized air through either the first pathway or the second pathway. However, the bypass valve can also be used to direct a portion of the pressurized air through the first pathway and a portion of the pressurized air through the second pathway. This partial diversion of the pressurized air can be used to cause only some of the substance that is contained within the receptacle 300 to be delivered into the air pathway. The bypass valve can be manually operable by the user 210, and/or can be controlled by the control system 110, in order to move the bypass valve between open and closed configurations, and to control the amount of partial diversion.

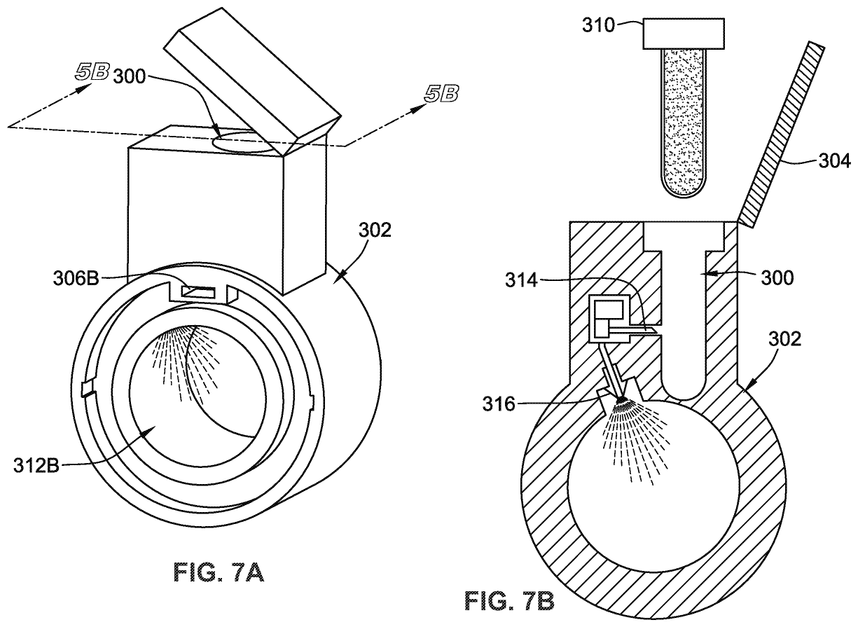

FIGS. 7A and 7B show a second implementation of the injection plug 302. The second implementation is generally similar to the first implementation, except that the injection plug 302 includes a separate injection mechanism 314 and nozzle 316 configured to deliver the substance into the air pathway. The injection plug 302 can generally be positioned in-line with the air pathway in any of the arrangements described above with reference to the injection plug 302 of FIGS. 6A and 6B. The injection plug 302 can include electrical contacts that can be used to heat the pressurized air, heat the substance, and/or electrically connect the injection plug 302 to the control system 110. The injection plug 302 also includes the movable cover 304 that controls access into the receptacle 300. Finally, the substance can be contained in a pod 310 that is received by the receptacle 300.

In the implementation of FIGS. 7A and 7B, the injection plug 302 has an air inlet 312A and an air outlet 312B formed from the open ends of the injection plug 302. However, the receptacle 300 is not directly open to the air pathway via apertures as in FIGS. 6A and 6B, but instead is fluidly coupled to the air pathway using a different mechanism or structure, such as via the injection mechanism 314 and the nozzle 316. The end of the nozzle 316 thus acts as the outlet of the receptacle 300 in the implementation of FIGS. 7A and 7B, while the receptacle 300 itself generally does not have an inlet via which pressurized air flows into the receptacle. The injection mechanism 314 is configured to pierce the pod 310 containing the substance to fluidly connect the pod 310 to the air pathway. The nozzle 316 is configured to control the release of the substance into the air pathway. The injection mechanism 314 can be a needle or other sharp object that is used to pierce the pod 310 and allow the substance to be delivered into the air pathway. The control system 110 is configured to control the injection mechanism 314 and the nozzle 316 so as to selectively deliver the substance into the air pathway. However, the injection mechanism 314 and/or the nozzle 316 could additionally or alternatively be manually operable by the user 210. In this implementation, the injection plug 302 again acts as a nebulizer that can aerosolize the substance.

In still other implementations, the nozzle 316 is not controlled by the user 210 or the control system 110. In these implementations, once the pod 310 is pierced by the injection mechanism 314, the substance in the pod 310 is automatically released into the air pathway through the nozzle 316. In these implementations, the injection mechanism 314 can be controlled by the user 210 or the control system 110 to pierce the pod 310 at a desired time, or the injection mechanism 314 can be configured to automatically pierce the pod 310 when the pod 310 is inserted into the receptacle 300. In these implementations or any other implementations, the substance can be a solid, liquid, or a gas.

Referring now to FIGS. 8A and 8B, the receptacle 300 can also be disposed within the housing 123 of the respiratory device 122, instead of being located external to the respiratory device 122 in a separate and distinct injection plug 302. As shown in FIGS. 8A and 8B, the housing 123 can define a cover 604 that is similar to cover 304 of the injection plug 302. Generally, all of the features of the cover 304 of the injection plug 302 can be present in the cover 604 of the housing 123 of the respiratory device 122. The cover 604 thus controls access to the receptacle 300 inside the housing 123, and can be controlled by the control system 110 and/or can be manually operable by the user 210. The receptacle 300 is configured to receive a pod 310 that contains the substance. In this implementation, the pod 310 is thus disposed within the housing 123 of the respiratory device 122 when the receptacle 300 receives the pod 310.

In the implementation illustrated in FIGS. 8A and 8B, the internal structure of the respiratory device 122 that forms the air pathway includes an injection plug 302, in which the receptacle 300 is formed. In this manner, the implementation illustrated in FIGS. 8A and 8B generally includes all of the features discussed herein with respect to the implementation in FIGS. 5A-7B. However, in some implementations, the receptacle 300 could simply be a portion of or all of the internal structure of the respiratory device 122 that forms the air pathway (e.g., tubing), without any separate injection plug 302.

The receptacle 300 in FIGS. 8A and 8B is generally similar to the receptacle 300 in FIGS. 6A-7B. The receptacle 300 can be positioned in-line with the air pathway inside the respiratory device 122 such that the pressurized air flowing through the receptacle 300 causes the substance to be delivered into the air pathway. In this implementation, the receptacle 300 is formed with apertures 309 similar to the receptacle 300 in FIGS. 6A and 6B, and has an inlet 312A and an outlet 312B that are in fluid communication with the air pathway. The receptacle 300 can alternatively have only an outlet 312B that is in fluid communication with the air pathway. In this implementation, the receptacle 300 includes an injection mechanism 314 and nozzle 316, which can be the same as or similar to the injection mechanism 314 and nozzle 316 of the receptacle 300 of FIGS. 7A and 7B.

FIG. 8B shows one location of the receptacle 300 within the housing 123 of the respiratory device 122. In the illustrated implementation, the respiratory device 122 includes a blower motor 250 with a motor inlet 252 and a motor outlet 254. The motor inlet 252 is in fluid communication with the atmosphere through the air inlet 180 of the respiratory device 122. The motor outlet 254 is in fluid communication with the interior of the humidification tank 129. During operation of the respiratory device 122, the blower motor 250 causes air to flow through the motor and through the humidification tank 129.

The blower motor 250 is electrically connected to a control board 256. In some implementations, the control board 256 contains the processor 112, and thus forms the control system 110. In other implementations, the control system 110 is separate from the control board 256. In these implementations, the control board 256 includes a separate processor and a communication interface to allow the control board 256 to communicate with the control system 110. The respiratory device 122 further includes a power converter 258 electrically connected to the control board 256. The power converter 258 includes a receptacle that opens to the exterior of the respiratory device 122 that allows the respiratory device 122 to be connected to an external power source, such as an electrical outlet via an external electrical cable. The power converter 258 powers the blower motor 250, the control board 256, and any other electronic components of the respiratory device 122.

While FIG. 8B shows the receptacle 300 positioned between the humidification tank 129 and the air outlet 190 of the respiratory device 122, the internal receptacle 300 can be positioned elsewhere along the air pathway. For example, the receptacle 300 could be positioned between the air inlet 180 and the blower motor 250, such that the inlet 312A of the receptacle 300/injection plug 302 is in fluid communication with the air inlet 180 and the outlet 312B of the receptacle 300/injection plug 302 is in fluid communication with the blower motor 250. In another example, the receptacle 300 could be positioned between the blower motor 250 and the humidification tank 129, such that the inlet 312A is in fluid communication with the blower motor 250 and the outlet 312B is in fluid communication with the humidification tank 129.

Referring generally to the various receptacles 300 illustrated in FIGS. 5B-8B, the receptacle 300 is generally separate and distinct from the humidification tank 129, whether the receptacle 300 is formed as part of the injection plug 302 or is located within the housing of the respiratory device 122. Thus, while the humidification tank 129 is configured to deliver water vapor into the air pathway and humidify the pressurized air, the receptacle 300 is distinct from the humidification tank 129. As noted and shown herein, in some implementations, the receptacle 300 is in fluid communication with a point along the air pathway that is downstream from the humidification tank 129 and between the humidification tank 129 and the user 210. However, in other implementations, the receptacle 300 can be in fluid communication with the air pathway at a point upstream from the humidification tank 129.

In addition, while the implementation of the receptacle 300 having the injection mechanism 314 and the nozzle 316 is described as being formed so that the pressurized air does not flow through the receptacle 300, it is contemplated that the receptacle 300 could have an inlet and an outlet, while still utilizing the injection mechanism 314 and the nozzle 316 to release the substance into the air pathway. Thus, while the pressurized air may generally flow into and through the receptacle 300, it is the operation of the injection mechanism 314 and the nozzle 316 that operate to release the substance into the air pathway.

Other mechanisms can also be used to assist or cause the substance to be delivered into the air pathway. In some implementations, the receptacle 300 includes a membrane that regulates the delivery of the substance into the air pathway. In these implementations, the membrane can act as a timed release mechanism to ensure that an appropriate amount of the substance is delivered into the air pathway at an appropriate time. In other implementations, the receptacle 300 includes a barrier that is configured to break down in response to the pressurized air being directed to the airway of the user 210. For example, the barrier could be configured to break down due to the force of the pressurized air flowing through the receptacle 300, thereby releasing the substance into the air pathway. In another example, the barrier is configured to dissolve when the pressurized air is humidified prior to being directed to the air pathway.

In still other implementations, the packaging of the substance itself forms a membrane or dissolvable barrier, instead of the membrane or dissolvable barrier being located in the receptacle 300. In these implementations, when the substance is inserted into the receptacle 300, the packing of the substance regulates the release of the substance into the air pathway, as described above. The substance could also be inserted into the air pathway at any point within the system without the use of a separate receptacle 300, such as being inserted directly into the humidification tank 129.

In some implementations, the receptacle 300 can contain at least two different substances. The receptacle 300 can be configured to maintain the two substances separately, and allow the substances to be mixed together when needed.

Referring to FIG. 9, a method 700 of delivering a substance into an air pathway of a user using a respiratory system (such as respiratory system 120) is illustrated. A memory device (such as memory device 114 of system 100) can be used to store machine-readable instructions and any type of data utilized in the steps of method 700. A control system (such as control system 110) can be used to execute the machine-readable instructions to cause the steps of method 700 to be performed. One or more of the steps of the method 700 described herein can be implemented using the system 100 (FIG. 1), and are described using the various components of the system 100. However, it is understood that the method 700 can be performed by appropriate systems other than system 100.

Step 702 of the method 700 includes receiving the substance in the receptacle 300. The receptacle 300 can be any of the implementations of the receptacle 300 described herein. The receptacle 300 can be formed as part of an external injection plug 302, or can be disposed within the housing 123 of the respiratory device 122. The receptacle 300 can be positioned in-line with the air pathway such that air flowing through the receptacle 300 causes the substance to be delivered into the air pathway, or the receptacle 300 can be configured to inject the substance into the air pathway. The receptacle 300 can directly receive the substance, or can receive the substance within the pod 310. Step 704 of the method 700 includes receiving physiological data from the one or more sensors 130. As noted herein, any number of the sensors 130 can be included as part of the respiratory system 120.

Step 706 of the method 700 includes determining, using the control system 110, one or more sleep-related parameters based at least in part on the received physiological data. As discussed herein, a large variety of sleep-related parameters can be determined based at least in part on the physiological data. The sleep-related parameters include a sleep score, a flow signal, a respiration signal, a respiration rate, an inspiration amplitude, an expiration amplitude, an inspiration-expiration ratio, a number of events per hour, a pattern of events, a stage, pressure settings of the respiratory device 122, a heart rate, a heart rate variability, movement of the user 210, temperature, EEG activity, EMG activity, arousal, snoring, choking, coughing, whistling, wheezing, or any combination thereof. Step 706 can also include receiving other data, such as feedback data related to prior uses of the substance and/or the respiratory device 122 by the user 210, or location data related to the location of the user or the ambient environment surrounding the user.

Finally, step 708 of the method 700 includes modifying the delivery of the substance into the air pathway based at least in part on the sleep-related parameters and/or the other data. The delivery of the substance into the air pathway can be modified in a variety of different ways, including by initiating the delivery of the substance into the air pathway, ending the delivery of the substance into the air pathway, modifying the amount of the substance that is currently being delivered into the air pathway, modifying a quantitative characteristic of the delivery, or modifying a temporal characteristic of the delivery (e.g., changing the rate at which the substance is delivered, starting and/or finishing at different times, continuing for different time periods, changing the time distribution or characteristics of the delivery, changing the amount distribution, independently of the time distribution, etc. The independent time and amount variation ensures that, apart from varying the frequency of substance release, one can vary the amount of substance released each time. So one can end up with different combination of release frequencies and release amount (e.g., higher frequency but lower release amount, higher frequency and higher amount, lower frequency and higher amount, lower frequency and lower amount, etc.). Generally, any parameters associated with the delivery of the substance in the air pathway can be modified, and multiple modifications can also be undertaken generally at the same time or step.

For example, the substance may cause the user 210 to have difficulty falling asleep. The sleep-related parameters could indicate that the user 210 is taking longer to fall asleep, or the user 210 could manually indicate that they are having trouble falling asleep. The control system 110 can then cause the substance to be injected into the air pathway at a later time, after the user 210 has fallen asleep.

In one implementation, the sleep-related parameters include a respiration rate, and the delivery of the substance into the air pathway is initiated if the respiration rate is above or below a first predetermined threshold respiration rate, or ended if the respiration rate is above or below a second predetermined threshold respiration rate different than the first predetermined threshold respiration rate. The substance can be configured to increase the respiration rate of the user 210, in which case delivery is initiated if the respiration rate of the user 210 is too low, or ended if the respiration rate of the user 210 is too high or is appropriate. The substance could also be configured to decrease the respiration rate of the user 210, in which case delivery is initiated if the respiration rate of the user 210 is too high, or ended if the respiration rate of the user 210 is too low or is appropriate.

In another implementation, the sleep-related parameter is a heart rate of the user 210, and the delivery of the substance into the air pathway is initiated if the heart rate is above or below a first predetermined threshold heart rate, or ended if the heart rate is above or below a second predetermined threshold heart rate different than the first predetermined threshold heart rate. The substance can be configured to increase the heart rate of the user 210, in which case delivery is initiated if the heart rate of the user 210 is too low, or ended if the heart rate of the user 210 is too high or is appropriate. The substance could also be configured to decrease the heart rate of the user 210, in which case delivery is initiated if the heart rate of the user 210 is too high, or ended if the heart rate of the user 210 is too low or is appropriate.

In a further implementation, the sleep-related parameters are used to determine an effectiveness of the substance, and to modify the delivery of the substance into the air pathway based at least in part on the effectiveness. Generally, the substance can be a medicament, and the sleep-related parameters can be used to determine how effectively the medicament is treating the disease or ailment of the user 210. The amount of the medicament being delivered into the air pathway could be increased in a larger dose is needed, or ended if the medicament is not effective.

In still another implementation, the other data is location data associated with the location of the user 210. The location data can in this case be defined to also include environmental data about the ambient environment surrounding the respiratory device 122 and/or the user 210 (e.g., the user 210's immediate surroundings such as their bedroom), or data about the user 210's larger surroundings, such as the user 210's house, neighborhood, etc. The location data could include sound or air pressure data in the user 210's bedroom (and/or other room(s) where the respiratory device 122 or any components of system 100 are located). This data, as well as device data, such as, for example, airflow data obtained from the flow rate sensor 134 of respiratory device 122, can be analyzed to determine whether any amount of the supplied pressurized air has escaped the user interface 124 or the conduit 126 or whether any amount of the portion of the substance has escaped the user interface 124 or the conduit 126. If some of the air has escaped, the control system 110 can end the delivery of the substance into the air pathway, or reduce the amount of the substance being delivered into the air pathway. The control system 110 could additionally or alternatively adjust the pressure of the pressurized air being supplied to the air pathway, for example by increasing the pressure to account for the leak in the air pathway. The location data can also include other identifying information such as geographic coordinates; weather data about the user 210's location (e.g., temperature, humidity, precipitation, wind); pollution data; or generally any other data about the user 210's surroundings that may be beneficial (the temperature, sound/noise level and light intensity in the room, a chemical analysis of the air in the room, etc.).

The control system 110 can utilize the location data to generate a notification for the user 210. The notification can be transmitted to the user 210 (for example, via the external device 170), and/or recorded (e.g., by the control system 110 and the memory device 114). For example, the control system 110 can generate a notification if it is detected that air is leaking from the air pathway. The notification could suggest to the user 210 a modification to the respiratory device 122, the user interface 124, or the conduit 126, in order to reduce or end the leak. The control system 110 could also generate an alert if the leak is detected (and is unintentional), and delay the delivery of the substance into the air pathway if the leak is detected (and is unintentional) until the leak diminishes or stops.

In still other implementations, the other data is feedback data. The feedback data can be associated with a prior use of or experience with the substance by the user 210, and can be used to modify the delivery of the substance into the air pathway to take into account the user 210's experience during past uses of the substance. For example, if the feedback data indicates that the user 210 reacted to a substance in a specific way, the feedback data can be used to prevent that substance from being released into the air pathway, to ensure that an appropriate amount of the substance (e.g., a smaller amount of the substance) is released into the air pathway, or to ensure that the release has optimized dynamics (e.g., is sufficient in amount and flow properties to reach the user). In another example, the user can indicate that the scent of the substance was too strong, that the substance was delivered too quickly, or that the effect of medication lingered for too long. The control system 110 can then ensure that the amount of the substance released into the air pathway is reduced, such that the substance release period is prolonged or that the time and/or amount distribution for the release events is suitably amended.

In some implementations, the user 210 utilizes the external device 170 to input the feedback data. For example, the external device 170 can be a smart phone, and the user 210 can enter information related to past uses of the system via the external device 170. The user 210 can also provide the feedback data in other ways (e.g., via a smart speaker). The feedback data can be objective (e.g., numerical data about the user 210's reaction to the substance) or subjective (e.g., the user 210's feelings about the substance, estimate of how the user feels before and after a release sequence, etc.) In some implementations, the physiological data and/or the sleep-related parameters can also be used as feedback data indicative of prior uses or experiences with the substance.

In an additional implementation, the other data is related to whether the user 210 is currently wearing the user interface 124, what type of interface 124 the user is currently wearing, whether a treatment algorithm is running on the respiratory device, etc. In this implementation, the control system 110 can analyze this data to determine whether the user 210 is currently wearing the user interface 124. If the control system 110 determines that the user 210 is not wearing the user interface 124, the control system 110 can generate and/or transmit an alert indicating that the user is not wearing the user interface 124. The control system 110 can also determine what type of interface 124 is currently being used, and adjust the delivery of the substance into the air pathway based at least in part on what type of interface 124 is being used. For example, if the type of user interface can affect the amount of released substance that is passed into the user 210's airway(s), the amount of the substance that is being delivered into the air pathway may be adjusted based at least in part on what type of interface 124 is being used.

In another implementation, the one or more sleep-related parameters includes the user 210's breathing pattern. The user 210's breathing pattern is indicative of when the user is inhaling and when the user is exhaling. In this implementation, the control system 110 is configured to cause the substance to be delivered into the air pathway only when the user is inhaling. If the substance is delivered into the air pathway when the user exhales, some amount of the substance can be forced out of the respiratory system (for example via vents in the respiratory device 120 or other components), instead of being delivered into the user's airway. In implementations where the injection plug 302 includes a bypass valve, the control system 110 can close the bypass valve when the user is inhaling (or right before the user begins to inhale), so that the flow of pressurized air from the respiratory device 120 will flow through the receptacle 300 containing the substance. The control system 110 can open the bypass valve when the user is exhaling (or right before the user begins to exhale), so that the air flowing from the user's exhale bypasses the receptacle 300. In implementations where the receptacle includes an injection mechanism 314 and a nozzle 316, the control system 110 can control the nozzle 316 to deliver the substance into the air pathway when the user is inhaling, and to stop delivering the substance into the air pathway when the user is not inhaling. In another implementation, the user 210 can actively cause the substance to be delivered into the air pathway at a certain time. For example, the user 210 can trigger the release of the substance from the receptacle when the user 210 is ready to inhale.

As described herein, the control system 110 is configured to receive a variety of other data that is not direct physiological data from the sensors 130. The other data can be received via the external device 170, which can receive input from the user, or could be derived from additional sensors that generate and transmit the data. The other data can also be received via one or more of the sensors 130, even if the other data is not direct physiological data about the user 210. For example, the sensors 130 can be used to generate feedback data or environmental data, that while generated by the sensors 130, is not physiological data about the user 210. Generally, any of the physiological data, the other data, or any other information can be stored in the memory device 114.

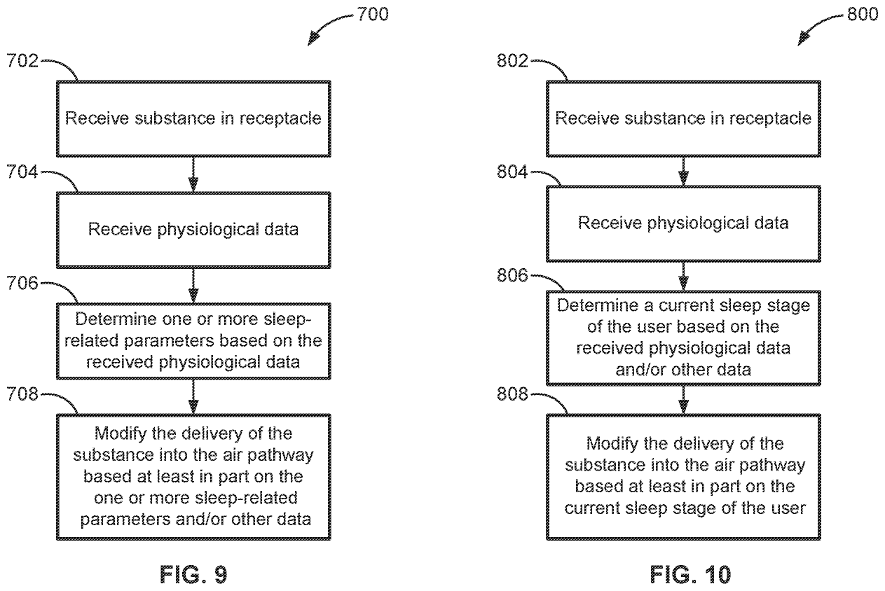

Referring to FIG. 10, a method 800 of delivering a substance into an air pathway of a user using a respiratory system (such as respiratory system 120) is illustrated. A memory device (such as memory device 114 of system 100) can be used to store machine-readable instructions and any type of data utilized in the steps of method 800. A control system (such as control system 110) can be used to execute the machine-readable instructions to cause the steps of method 800 to be performed. One or more of the steps of the method 800 described herein can be implemented using the system 100 (FIG. 1), and are described using the various components of the system 100. However, it is understood that the method 800 can be performed by appropriate systems other than system 100.

Step 802 of the method 800 includes receiving the substance in the receptacle 300. The receptacle 300 can be any of the implementations of the receptacle 300 described herein. The receptacle 300 can be formed as part of an external injection plug 302, or can be disposed within the housing 123 of the respiratory device 122. The receptacle 300 can be positioned in-line with the air pathway such that air flowing through the receptacle 300 causes the substance to be delivered into the air pathway, or the receptacle 300 can be configured to inject the substance into the air pathway. The receptacle 300 can directly receive the substance, or can receive the substance within the pod 310. The substance itself can be located within packaging. Step 804 of the method 800 includes receiving physiological data from the one or more sensors 130. As noted herein, any number of the sensors 130 can be included as part of the respiratory system 120.

Step 806 of the method 800 includes determining, using the control system 110, the current sleep stage of the user 210 within the sleep session. At this step, it is generally determined whether the user 210 is asleep, and if the user 210 is asleep, what stage of sleep the user is in (e.g., light sleep, deep sleep, REM sleep, etc.). This determination can be based at least in part on the received physiological data and/or other data. In some implementations, the physiological data is used to determine one or more of the sleep-related parameters, which can be used to determine the current stage of the sleep session. Finally, step 808 of the method 800 includes modifying the delivery of the substance into the air pathway based at least in part on the current stage of the sleep session. Modifying the delivery of the substance into the air pathway can involve any modification discussed herein, including those modifications discussed herein with respect to step 708 of method 700.

In one implementation, the control system 110 determines whether the user 210 has entered a predetermined or desired stage of the sleep session, and initiates the delivery of the portion of the substance into the air pathway, ends the delivery of the portion of the substance into the air pathway, modifies the amount of the substance being delivered into the air pathway, and/or modifies the frequency at which the substance is delivered. For example, the control system 110 can initiate the delivery of a medicament only once the user 210 has fallen asleep, or only once the user 210 has awakened after sleeping. In another example, the control system 110 can initiate the delivery of a medicament when the user 210 is initially awake at the beginning of the sleep session, and then reduce (in amount or frequency) or end the delivery of the medicament once the user 210 falls asleep.

In another implementation, the control system 110 can initiate the delivery of a first substance into the air pathway when the user 210 is in a first stage, and initiate the delivery of a second substance into the air pathway when the user 210 is in a second stage. The second substance can be delivered instead of or in addition to the first substance. In one example, the user 210 has a disease or ailment that can be treated while the user 210 is asleep. In this example, the first substance is a sleep aid that is delivered when the user 210 is awake, and the second substance is a medicament. In another example, the user 210 has a disease or ailment that preferably treated only while the user 210 is awake. In this example, the first substance is the medicament, and the second substance is configured to aid the user 210 in waking up that is only delivered when the user 210 is asleep.

In a further implementation, the control system 110 is configured to start and stop the delivery of the substance into the air pathway based at least in part on whether the user 210 is awake or asleep. In some cases, it is undesirable to deliver a medicament to the air pathway while the user 210 is asleep. Thus, if the control system 110 determines that the user 210 is awake the substance (which can be a medicament) can begin to be delivered into the air pathway. When the control system 110 determines at a later time that the user 210 is asleep, the system can end the delivery of the medicament into the air pathway. The control system 110 can also determine whether the user 210 is still asleep after a desired waking point, and initiate the delivery of the sleep inhibitor to aid the user 210 in waking up. Alternatively, the control system 110 can determine whether the user 210 is awake prior to a desired waking point, and initiate the delivery of a sleep aid to aid the user 210 in falling asleep.

In some implementations, the other data utilized by the control system 110 includes temporal data, such as an amount of time spent in the current stage of the sleep session. The modification of the delivery of the substance into the air pathway can be based at least in part on the temporal data. For example, if the time spent in the current stage is greater than or less than a threshold time, the control system 110 can initiate or end the delivery of the substance into the air pathway. If the current stage is a stage where the user 210 is awake, the delivery of a sleep aid or calming scents or aromas into the air pathway can be initiated if the user 210 is having difficulty falling asleep. If the current stage is a stage where the user 210 is asleep, the delivery of a consciousness-arousing compound (e.g., an anti-sleep aid) can be initiated if the user 210 is having difficulty waking up. In one example, if the user 210 has been in the current stage (such as being awake) for more than two hours, the substance (such as a sleep aid) could begin to be delivered into the air pathway. In another example, the delivery of the substance into the air pathway can be initiated based at last in part on the current time. In a further example, the system monitors the user 210 over a predetermined time period, determines whether the user 210 has been asleep or awake for at least a predetermined minimum amount of time within the time period, and only then initiates the delivery of the substance into the air pathway.

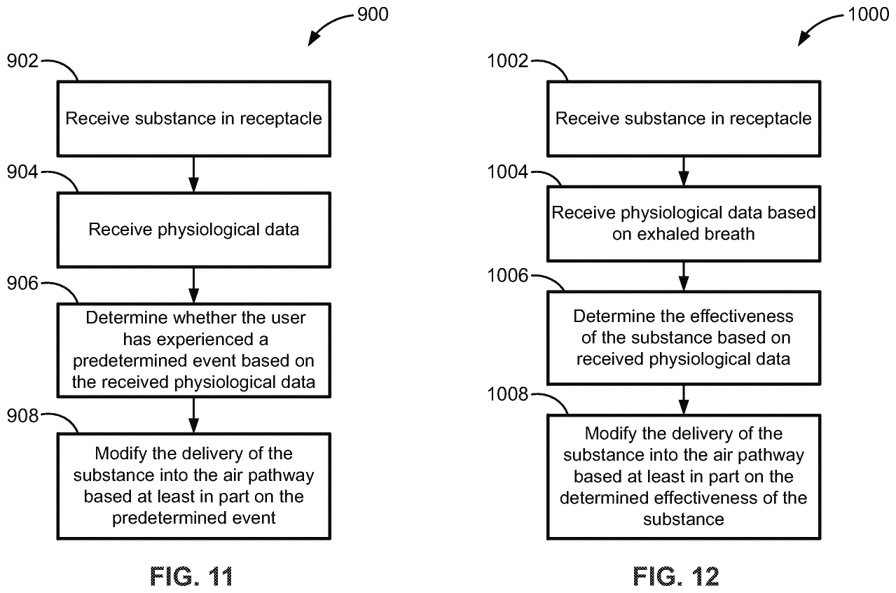

Referring to FIG. 11, a method 900 of delivering a substance into an air pathway of a user using a respiratory system (such as respiratory system 120) is illustrated. A memory device (such as memory device 114 of system 100) can be used to store machine-readable instructions and any type of data utilized in the steps of method 900. A control system (such as control system 110) can be used to execute the machine-readable instructions to cause the steps of method 900 to be performed. One or more of the steps of the method 900 described herein can be implemented using the system 100 (FIG. 1), and are described using the various components of the system 100. However, it is understood that the method 900 can be performed by appropriate systems other than system 100.

Step 902 of the method 900 includes receiving the substance in the receptacle 300. The receptacle 300 can be any of the implementations of the receptacle 300 described herein. The receptacle 300 can be formed as part of an external injection plug 302, or can be disposed within the housing 123 of the respiratory device 122. The receptacle 300 can be positioned in-line with the air pathway such that air flowing through the receptacle 300 causes the substance to be delivered into the air pathway, or the receptacle 300 can be configured to inject the substance into the air pathway. The receptacle 300 can directly receive the substance, or can receive the substance within the pod 310. Step 904 of the method 900 includes receiving physiological data from the one or more sensors 130. As noted herein, any number of the sensors 130 can be included as part of the respiratory system 120.

Step 906 of the method 900 includes determining, using the control system 110, whether the user 210 has experienced an adverse event. An adverse event is generally any event that affects the user 210 in an adverse manner. For example, an adverse event could be a breathing event due to a sleep disorder, e.g., the cessation (apnea) or reduction (hypopnea) in breathing in a user 210 with sleep apnea. The adverse event can also include general health-related events, such as asthma attacks, heart attack or other cardiac events, coughing, snoring, etc. In some implementations, the user 210 waking up at an undesirable time (such as the middle of the night) is an adverse event.

The determination of whether the user 210 has experienced an adverse event can be based at least in part on the received physiological data which are used to determine sleep-related parameters, or any other data received (e.g., non-physiological data). For example, data about the user 210's heart (such as a heart rate of the user 210) can be used to determine whether the user 210 has suffered a heart attack. In another example, data about the user 210's respiration (such as a respiration rate of the user 210) can be used to determine whether the user 210 has suffered an asthma attack. Finally, step 908 of the method 900 includes modifying the delivery of the substance into the air pathway based at least in part on whether the user 210 has experienced an adverse event. Modifying the delivery of the substance into the air pathway can involve any modification discussed herein, including those modifications discussed herein with respect to step 708 of method 700. Method 900 can also be utilized when the user 210 experiences any type of predetermined event, whether adverse or not.

In some implementations, the control system 110 detects that the user 210 has suffered or is suffering a heart attack, and can initiate the delivery of a medicament to treat the heart attack into the air pathway. Similarly, the control system 110 can detect that the user 210 has suffered or is suffering an asthma attack, and can initiate the delivery of a medicament to treat the asthma attack into the air pathway. Other adverse events can also be detected, such as a sleep apnea event, an event involving inflammation, or any event that causes the user 210 to cease breathing.

The control system 110 can also be configured to generate and/or transmit an alert (or cause an alert to be generated and/or transmitted) based at least in part on a determination that the user 210 has experienced an adverse event. For example, the control system 110 can alert an emergency services provider or an emergency contact when the user 210 experiences an adverse event.

In further implementations, the control system 110 can determine whether the user 210 undergoes a desired/expected motion pattern following the adverse event. Such a motion pattern can include one or more body movements of the user 210. If the user 210 has not undergone the desired motion pattern within a desired time period, the control system 110 can initiate the delivery of a medicament into the air pathway, and/or can contact an emergency services provider.

In still other implementations, it is determined whether the user experiences any type of predetermined event, whether adverse or not. The delivery of the substance into the air pathway can then be modified in response. In some implementations, the predetermined event can include the user 210 being asleep for less than a predetermined length of time, the user being asleep for greater than a predetermined length of time, the user being awake for less than a predetermined length of time, or the user being awake for greater than a predetermined length of time.

In one implementation, the receptacle is configured to receive a first substance and a second substance different than the first substance. In response to determining that the user has experienced a first type of predetermined event (which could be a first type of adverse event), the control system 110 causes the first substance to be delivered into the air pathway of the user 210. In response to determining that the user has experienced a second type of predetermined event (which could be a second type of adverse event), the control system 110 causes the second substance to be delivered into the air pathway of the user 210. This implementation allows the system 100 to be prepared for multiple different types of events the user 210 may experience while they sleep, such as both a heart attack and an asthma attack. This implementation also allows the system 100 to deliver a non-medicament (such as an aromatherapy compound), while still being prepared to deliver a medicament if the user experiences an adverse event (such as a heart attack). In still another implementation, the first substance is a medicament configured to treat a medical condition or event, and the second substance is configured to counteract a potential adverse response to the first substance.

In these implementations, the physiological data can be analyzed to determine the user 210's physiological response to the substance delivered into the air pathway. If the user 210 does not respond as strongly as desired, the amount of the substance being delivered into the air pathway can be increased. Conversely, of the user 210 responds more strongly than desired, the amount of the substance being delivered into the air pathway can be decreased. A summary of the user 210's response to the substance can also be prepared and transmitted to the user 210 or to a third party, such as a caregiver or a healthcare provider.

Referring to FIG. 12, a method 1000 of delivering a substance into an air pathway of a user using a respiratory system (such as respiratory system 120) is illustrated. A memory device (such as memory device 114 of system 100) can be used to store machine-readable instructions and any type of data utilized in the steps of method 1000. A control system (such as control system 110) can be used to execute the machine-readable instructions to cause the steps of method 1000 to be performed. One or more of the steps of the method 1000 described herein can be implemented using the system 100 (FIG. 1), and are described using the various components of the system 100. However, it is understood that the method 1000 can be performed by appropriate systems other than system 100.

Step 1002 of the method 1000 includes receiving the substance in the receptacle 300. The receptacle 300 can be any of the implementations of the receptacle 300 described herein. The receptacle 300 can be formed as part of an external injection plug 302, or can be disposed within the housing 123 of the respiratory device 122. The receptacle 300 can be positioned in-line with the air pathway such that air flowing through the receptacle 300 causes the substance to be delivered into the air pathway, or the receptacle 300 can be configured to inject the substance into the air pathway. The receptacle 300 can directly receive the substance, or can receive the substance within the pod 310.

Step 1004 of the method 1000 includes receiving physiological data from the one or more sensors 130 associated with the exhaled breath of the user 210 before, during, and/or after the portion of the substance has been delivered into the air pathway of the user 210. As noted herein, any number of the sensors 130 can be included as part of the respiratory system 120. In this implementation, at least one of the sensors 130 is positioned in an exhalation path of the user 210 (e.g., adjacent to a mouth or a nose of the user 210), and can be used to detect the presence and concentration of an analyte in the exhaled breath of the user 210.

Step 1006 of the method 1000 includes determining, using the control system 110, the effectiveness of the substance based at least in part on the physiological data. For example, the presence and concentration of the analyte can indicate whether a medicament that is being delivered to the user 210 is effective. In one example, the concentration of the analyte in the exhaled breath of the user 210 after the substance is delivered into the air pathway is used to determine the amount of the substance that has been absorbed by the user 210. The absorbed amount of the substance can be compared to the amount of the substance that was delivered into the air pathway of the user 210 to determine the effectiveness of the substance. In another example, the concentration in the analyte in the exhaled breath of the user prior to delivering the substance into the air pathway of the user 210 is compared to the analyte concentration in the exhaled breath of the user after delivering the substance into the air pathway of the user 210. This comparison can be used to determine the amount of the substance absorbed by the user 210.

Finally, step 1008 of the method 1000 includes modifying the delivery of the substance into the air pathway based at least in part on the determined effectiveness of the substance medicament. Modifying the delivery of the substance into the air pathway can involve any modification discussed herein, including those modifications discussed herein with respect to step 708 of method 700.

For example, if the substance is a medicament and the current amount is not effective, the control system 110 can increase the amount of the medicament that is delivered to the air pathway. The control system 110 can also end the delivery of the current medicament into the air pathway and initiate the delivery of a different medicament into the air pathway. In other implementations, the control system 110 can generate an alert if the medicament is not effective (for example following an adverse event).

In some implementations, the control system 110 is configured to compare the concentration of the analyte to a predetermined threshold to determine the effectiveness of the medicament. In some examples, the analyte is only present if the disease or ailment of the user 210 is not being treated, and thus a concentration larger than the threshold can indicate the medicament is not effective. Alternatively, the analyte may be a byproduct of the disease or ailment being treated by the medicament, in which case a concentration smaller than the threshold can indicate that the medicament is not effective.

In some implementations, determining the effectiveness of the medicament includes determining whether the user 210 has overdosed on the substance. This determination can be based at least in part on the physiological data indicating that one or more sleep-related parameters are above or below a desired threshold value. If the user 210 has overdosed on the medicament, the control system 110 can end the delivery of the medicament into the air pathway, and/or initiate the delivery of another substance into the air pathway that is configured to counteract the overdose.

As noted herein, the control system 110 can be configured to cause a notification to be sent in response to an overdose (or other adverse event). The notification could be sent to the user 210 and/or any desired third parties, such as family members, friends, emergency personnel (e.g., first responders, paramedics), or medical practitioner (e.g., the user 210's doctor). In general, with any of the methods described in FIGS. 9-12, the system 100 can generate a notification based at least in part on the physiological data or other data, and record and/or transmit the notification. The notification can be an indication of the effectiveness of the substance, adverse events caused by the substance, other adverse events unrelated to the substance, or generally any other phenomena or event that the user 210 or other desired third parties may want or need to be notified about.

According to some implementations of the present disclosure, the system 100 can be used to track cognitive function of the user 210 and/or detect cognitive decline in the user (e.g., a decrease in the user 210's cognitive function). An impaired sense of smell can be an early predictor of cognitive decline, such as, for example, future development of Alzheimer's disease, other types of dementia, and/or other neurodegenerative disease. The system 100 can be used to detect and monitor how the user 210 reacts to various scents and/or aromas injected into the air pathway of the user 210. One or more different scents can be injected by the system 100 in series and/or parallel for use in gauging a reaction of the user. Generally, the substance used with the system 100 for this purpose will have a specific aroma and/or scent that generally produces a detectable reaction in a person (e.g., a person with a standard cognitive function or a person without cognitive decline).

During and/or after the user 210 is exposed to the substance (e.g., before and/or during a sleep session with the respiratory device 122), the system 100 can receive and store physiological data and/or information indicative of the user 210's ability to detect or not detect the scent of the substance. In some implementations, one or more of the sensors 130 are used to generate such physiological data, which can then be transmitted to the control system 110 and stored in the memory device 114. The physiological data can include (i) respiration data, which can be used to determine parameters such as respiration rate, inspiration amplitude, expiration amplitude, inspiration-expiration ratio, or a respiration signal; (ii) cardiac data, which can be used to determiner parameters such as heart rate, heart rate variability, or blood pressure; (iii) data related to dilation of the user 210's pupils; (iv) electrical data associated with the user 210's brain, which can be used to model brain activity (e.g., brain waves) of the user 210; (v) electrical data associated with the user 210's skin, such as galvanic skin response, or (vi) any combination thereof.

The physiological data can be generated using any one or more of the sensors 130, such as the pressure sensor 132, the flow rate sensor 134, the temperature sensor 136, the a motion sensor 138, the microphone 140, the speaker 142, the acoustic sensor 141, the radio-frequency (RF) receiver 146, the RF transmitter 148, the RF sensor 147, the camera 150, the infrared (IR) sensor 152, the PPG sensor 154, the ECG sensor 156, the EEG sensor 158, the capacitive sensor 160, the force sensor 162, the strain gauge sensor 164, the EMG sensor 166, a galvanic skin response (GSR) sensor, or any other sensor.

The physiological data can be indicative of how the user reacts to the scent of the substance(s). For example, specific brain activity may occur when the user detects the scent. The electrical data associated with the user 210's brain can be used to monitor the user 210's brain activity when the substance is delivered into the air pathway. In another example, the cardiac data can be utilized to determine if there is any distinct change in the user 210's heart rate (for example a spike in the heart rate) when the substance is delivered into the air pathway. In still another example, the galvanic skin response of the user can change in response to the user 210 detecting the scent. In still other implementations, the control system 110 can use the data to determine sleep-related parameters, such as a sleep score, a number of respiration events per hours, a number of hours that the user is asleep during the sleep session, etc. These sleep-related parameters can also be used to determine how a user reacts to the scent of the substance. Furthermore, any or all of the various types of physiological data and resulting parameters may also be used to directly measure the cognitive functioning of the user 210.

In additional or alternative implementations, usually applied during a pre-sleep session, the control system can receive information from the user 210, for example via the external device 170. This information is related to the user 210's ability to smell the substance. In some implementations, the control system 110 is configured to ask the user 210 questions, such as through an application on the external device 170 (e.g., an app on a smartphone). The questions can be generally related to whether the user 210 is able to smell the substance that was injected into the air pathway. By providing answers to the questions, the user 210 provides feedback to the system indicative of their ability to detect the scent of various substances. The user 210 can input this information while the user 210 is wearing the user interface 124 and using the respiratory device 122 prior to falling asleep. The user 210 can additionally or alternatively input information once the user 210 has woken up, for example, after the sleep session has ended.

The system 100 can be used to monitor the user 210's ability to detect scents, as well as how strong the scent is to the user 210. This monitoring can be done in a variety of ways. For example, after delivery of a substance with a specific scent into the air pathway of the user 210, the user 210 can indicate what kind of scent they were able to detect (e.g., strawberry scent, flower scent, bubble gum scent, skunk scent, etc.). In some implementations, the control system 110 can ask the user (for example, via an application running on the external device 170) if they detected a scent, what kind of scent they detected, how many scents they detected, how long they detected the scent for, etc. The control system 110 could ask the user 210 to simply enter what kind of scent they detected, or ask the user 210 to choose from a variety of different options (e.g., via a multiple choice prompt). In some implementations, a different substance is used for successive sleep sessions, so as to deliver a different scent to the user 210 one night to the next.

The delivery of the substance for the purpose of cognitive testing may be different than the purpose associated with medication via aroma delivery (e.g., aromatherapy). In one example, delivering the substance for cognitive testing may include delivering a discrete amount of the substance to the user periodically, with a sufficient amount of time (e.g., one minute, five minutes, thirty minutes, one hour, two hours, three hours, four hours, five hours, etc., or any other amount of time) between the deliveries to collect physiological data or to receive feedback from the user. The amounts can progressively increase or decrease, depending, for example, on the response of the user 210. For instance, if the user 210 reacts strongly, either physiologically and/or via direct response to a question, the amounts may be progressively reduced to test the limit of the user 210's sensitivity. In contrast, when the user does not detect the substance, the amount can be progressively increased until an appreciable response is detected and/or received. The threshold where the user loses/gains sensitivity to the substance may provide a useful information of the cognitive state of the user.

In some implementations, the substance is continually delivered into the air pathway of the user 210 for substantially all of the sleep session, including when the user 210 is asleep. If the user 210 is able to detect the scent, it is likely that the user 210 will remember that scent once the user 210 wakes up. The user 210 can then provide that information to the control system 110. In still other implementations, the user 210 can initiate a testing procedure (for example using the external device 170) that prompts the user 210 for a variety of information related to detecting scents.

In some implementations, the control system 110 can also utilize non-physiological data. For example, the control system 110 can analyze the user 210's medical records to determine if the user 210 or a family member has any history of cognitive disease, or if there is any other information that can assist in determining whether the user 210 may be suffering from decreased cognitive function. For example, the user 210's medical records could indicate that there is some other reason that the user 210 is not able to detect scents as well as might be expected, for example, due to a medication that the user 210 is taking or another disease or condition that the user 210 is suffering from. In another example, medication that the user 210 is currently taking can indicate that the user 210 is suffering from cognitive decline. In still other implementations, the user 210's medical records may indicate directly that the user is suffering from cognitive decline or other neurodegenerative disease. The control system 110 can use this information to assist in determining whether the user is suffering from decreased cognitive function. The user 210's age can also be taken into account to assess any contribution that general deterioration of cognitive functions with age may have on the detected results. Absolute or relative values of the user 210's detected state of cognitive functions can be used. For example, the user 210's test results can be compared to a baseline associated with user 210's earlier test results, with test results statistically derived for relevant population group, etc.

In still other implementations, the non-physiological data includes subjective information that can be inputted directly by the user 210. The information can include information about the user 210's current medical status (e.g., how are they feeling, what kinds of conditions or diseases do they have), information related to any medication currently being taken by the user 210, information about the user 210's ability to detect the scent of a substance, information about the user 210's cognitive functioning, etc. In some implementations, the information includes an indication of the user 210's ability to detect the scent of the substance that is unrelated to the user 210's cognitive functioning level. In other implementations, the information includes an indication of the user 210's cognitive functioning level that is unrelated to the user 210's ability to detect the scent of the substance. Generally, the subjective information can include any relevant information that can be supplied by the user 210, or by other persons or sources.

The control system 110 can also utilize data from games or other activities undertaken by the user 210 indicative of the user 210's cognitive function. For example, the system 100 could deploy through the external device 170 a variety of different memory tests, puzzles, quizzes, etc., for the user 210 to complete. The results of these activities (e.g., how fast the user completed the test, how accurate the user was when taking the test, etc.) can be used by the control system 110 in its analysis of the user 210's cognitive function. The user 210 could also input results from these memory tests, puzzles, quizzes, etc., via the external device 170 and/or by another method.

The control system 110 is configured to analyze the physiological data, the information provided by the user 210, and any other data to generate one or more metrics related to the user 210's ability to detect scents, which in turn can be used to determine a level of cognitive function of the user 210. The system 100 can generate and store a time series of the one or more metrics to track the user 210's cognitive function over time (e.g., over a period of minutes, hours, days, weeks, months, years, decades, etc.). In some implementations, values of the one or more metrics satisfying one or more threshold values (e.g., being greater than, less than, or equal to the one or more threshold values) can indicate that the user 210 has decreased cognitive functioning, or is otherwise at risk of developing a cognitive disease in the future. In other implementations, the system 100 can perform a variety of different statistical operations on the metrics to determine whether the user has decreased cognitive functioning, or is otherwise at risk of developing a cognitive disease in the future. In some implementations, the control system 110 utilizes running averages over a time period to analyze the user 210's cognitive function. Using running averages over a time period can minimize the effect that unrelated temporary events may impact the values of one or more metrics.

In some implementations, the system 100 first establishes a baseline or initial level of cognitive functioning of the user 210. The system 100 can then subsequently compare the real-time level of cognitive functioning of the user 210 to the baseline level, in order to determine that the user 210's level of cognitive functioning has decreased below the baseline level.

When the system 100 determines that the user 210's cognitive function has decreased, determines and/or finds an indication of potential cognitive decline or that the user 210 is at risk for developing a cognitive disease in the future, or generally determines that the cognitive functioning of the user 210 satisfies some threshold (e.g., the cognitive functioning level has dropped below some baseline cognitive functioning level, the amount of decrease in the cognitive functioning level has exceed a threshold decrease, etc.), a variety of different actions can be taken. In some implementations, the system 100 can notify the user 210 and suggest additional diagnostic tests to better diagnose the user 210's cognitive function. In additional or alternative implementations, the system 100 can prompt the user 210 to input additional information about what types of events and/or other phenomena might be impacting the user 210's sense of smell. This information can help the system 100 avoid false positives.

In still other implementations, the control system 110 is configured to cause a notification to be transmitted to one or more third parties (e.g., relatives, parents, children, friends, doctors, care providers, etc.) in response to determining that the user 210 is suffering from decreased cognitive function or developing a cognitive function-impacting disease. For example, the control system 110 can transmit a notification indicating this determination to a health care provider (such as a doctor or a nurse), emergency services personnel, a family member, a friend, a health insurance provider, etc.

The control system 110 can implement a training or learning process (e.g., training a cognitive decline detection algorithm, training a smell detection algorithm, etc.) to more accurately determine whether the user 210 is suffering from cognitive decline, and determine which types of scents are more indicative of cognitive functioning for a specific user 210 or group of users or all users generally. Generally, the control system 110 can compare any type of received data (physiological, non-physiological, subjective, etc.) to identify the values of the various metrics or trends in the various metrics that can indicate a decline in the user 210's cognitive function. The control system 110 can thus rule out false positives, or other trends or values in the metrics that might be unrelated to the user 210's cognitive functioning. This feature can be used to determine which types of substances/scents most accurately reflect the user 210's cognitive functioning. For example, if there is a certain scent that the user is unable to detect (which could be indicated by physiological data and/or non-physiological data), the control system 110 can analyze any other data (such as the subjective information) to see if there is other any indication of cognitive decline. If there is no indication of cognitive decline from such an analysis (or an indication of level or even improved cognitive functioning), the control system 110 can ensure that that substance/scent is no longer used to test that specific user 210's cognitive functioning, because the system 100 has learned that such a substance/scent will not provide accurate or reliable results.

Because the delivery of the substance into the air pathway can be controlled and based on physiological data and/or sleep-related parameters, the system 100 and methods 700-1000 aid in ensuring that the correct dosage of the substance is delivered into the air pathway, either as a single bolus or over a predetermined time period. Potential errors in dosage amounts due to manual control (e.g., asthma inhalers) can be avoided.

Generally, any of methods 700, 800, 900, and 1000 can be implemented using a system having a control system with one or more processors, and a memory storing machine readable instructions. The controls system can be coupled to the memory, and methods 700, 800, 900, and 1000 can be implemented when the machine readable instructions are executed by at least one of the processors of the control system. Methods 700, 800, 900, and 1000 can also be implemented using a computer program product (such as a non-transitory computer readable medium) comprising instructions that when executed by a computer, cause the computer to carry out the steps of methods 700, 800, 900, and 1000.

One or more elements or aspects or steps, or any portion(s) thereof, from one or more of any of claims 1-150 below can be combined with one or more elements or aspects or steps, or any portion(s) thereof, from one or more of any of the other claims 1-150 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1-126. (canceled)

127. A respiratory therapy system comprising:
a respiratory device configured to supply pressurized air to an airway of a user via a user interface coupled to the respiratory device via a conduit, the respiratory device, the conduit, and the user interface forming an air pathway; and
an injection plug including an inlet, an outlet, a bypass valve, and a receptacle that includes a substance therein, the injection plug being fluidly coupled to the air pathway so that the inlet, the receptacle, and the outlet are in indirect or indirect fluid communication with the air pathway and at least a portion of the pressurized air delivered to the airway of the user is configured to enter the injection plug via the inlet, pass through the receptacle, and exit the injection plug through the outlet to cause at least a portion of the substance to be delivered into the air pathway,
wherein the bypass valve is fluidly connected to the receptacle and is configured to cause the supplied pressurized air to selectively pass through the receptacle or bypass the receptacle.

128. (canceled)

129. The system of claim 127, wherein the injection plug is disposed outside of a housing of the respiratory device.

130. The system of claim 129, wherein the injection plug is configured to be fluidly coupled between the respiratory device and the conduit, between the conduit and the user interface, or to an air inlet of the respiratory device.

131. The system of claim 127, wherein the receptacle is disposed within a housing of the respiratory device.

132. The system of claim 131, wherein the housing of the respiratory device includes a cover movable between a first position and a second position to control access to the receptacle.

133. The system of claim 132, wherein when the cover is in the first position, the receptacle is open and able to receive the substance therein, and when the cover is in the second position, the receptacle is closed and unable to receive the substance therein.

134. The system of claim 131, wherein the inlet of the receptacle is fluidly coupled to a blower motor of the respiratory device, and wherein the outlet of the receptacle is fluidly coupled to an air outlet of the respiratory device.

135. The system of claim 127, wherein the injection plug includes a cover movable between a first position and a second position to control access to the receptacle.

136. The system of claim 135, wherein when the cover is in the first position, the receptacle is open and able to receive the substance therein, and when the cover is in the second position, the receptacle is closed and unable to receive the substance therein.

137-138. (canceled)

139. The system of claim 127, wherein the bypass valve has a first pathway that bypasses the receptacle, and a second pathway fluidly coupled to the inlet of the receptacle.

140-143. (canceled)

144. The system of claim 127, wherein the respiratory device includes a heater configured to heat the portion of the substance and cause the portion of the substance to evaporate.

145. The system of claim 127, wherein the respiratory device includes a heater and a humidification tank fluidly connected to the air pathway, the humidification tank being configured to be heated by the heater to humidify the supplied pressurized air.

146. The system of claim 145, wherein the humidification tank is configured to aid in delivering water vapor into the air pathway.

147-149. (canceled)

150. The system of claim 127, wherein the receptacle is separate and distinct from the respiratory device.

151. A respiratory therapy system comprising:
a respiratory device configured to supply pressurized air to an airway of a user via a user interface coupled to the respiratory device via a conduit, the respiratory device, the conduit, and the user interface forming an air pathway; and
a receptacle including a substance therein, the receptacle being fluidly coupled to the air pathway so that the substance can be delivered into the air pathway,
wherein the receptacle includes an injection mechanism and a nozzle, the injection mechanism being configured to pierce a pod containing the substance, the nozzle being configured to release the portion of the substance into the air pathway.

152. The system of claim 151, wherein the injection mechanism and the nozzle are controllable to selectively release the portion of the substance into the air pathway.

153. The system of claim 151, wherein the injection mechanism and the nozzle are configured to aerosolize the portion of the substance.

154. The system claim 151, wherein the receptacle, injection mechanism, and the nozzle form a nebulizer.

155. A respiratory therapy system comprising:
a respiratory device configured to supply pressurized air to an airway of a user via a user interface coupled to the respiratory device via a conduit, the respiratory device, the conduit, and the user interface forming an air pathway; and
a receptacle including a substance therein, the receptacle being fluidly coupled to the air pathway so that the substance can be delivered into the air pathway,
wherein the receptacle includes a membrane such that the portion of the substance flows across the membrane when the portion of the substance is caused to be delivered into the air pathway, the membrane regulating the delivery of the portion of the substance into the air pathway.

156. A respiratory therapy system comprising:
a respiratory device configured to supply pressurized air to an airway of a user via a user interface coupled to the respiratory device via a conduit, the respiratory device, the conduit, and the user interface forming an air pathway; and
a receptacle including a substance therein, the receptacle being fluidly coupled to the air pathway so that the substance can be delivered into the air pathway,
wherein the receptacle includes a barrier configured to prevent the substance from being delivered into the air pathway, and barrier being configured to break down in response to the pressurized air being directed to the airway of the user such that the substance is delivered into the air pathway.

157. The system of claim 156, wherein the pressurized air is configured to be humidified, and wherein the barrier is configured to dissolve in response to the humidified pressurized air being directed to the airway of the user.
